(19) United States
(12) Patent Application Publication
(10) Pub. No.: US 2003/0147842 A1
(43) Pub. Date: Aug. 7, 2003

(54) Cosmetic compositions containing a particular aminosilicone and a conditioner, and uses thereof

(76) Inventors: Serge Restle, Saint-Prix (FR); Jonathan Gawtrey, Boulogne (FR); Sandrine Decoster, Saint Gratien (FR); Mireille Maubru, Chatou (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/290,226

(22) Filed: Nov. 8, 2002

(30) Foreign Application Priority Data
Nov. 8, 2001 (FR) 01 14480
Nov. 8, 2001 (FR) 01 14482

(51) Int. Cl. A61K 7/06; A61K 7/11

(52) U.S. Cl. 424/70.122

(57) ABSTRACT

A novel cosmetic composition comprising, in a cosmetically acceptable medium, at least one aminosilicone comprising at least one aminoethylimino(C₄-C₈)alkyl group as defined herein and at least one conditioner, being able to afford, for example, at least one improved cosmetic property (such as lightness, disentangling, volume and sheen) and/or at least one of persistent and remanent effects, as well as uses of the composition, such as for washing and/or conditioning keratin materials such as the hair or the skin.

Disclosed herein is a novel cosmetic composition comprising, in a cosmetically acceptable medium, at least one particular aminosilicone and at least one conditioner, as well as uses of this aminosilicone.

It is well known that hair that has been sensitized (i.e. damaged and/or embrittled) to varying degrees by the action of atmospheric agents or by the action of mechanical or chemical treatments, such as dyeing, bleaching and/or permanent-waving, may often be difficult to disentangle and to style, and may lack softness.

It has already been recommended, in compositions for washing or caring for keratin materials such as hair, to use conditioners, such as cationic polymers or silicones, to facilitate the disentangling of the hair and to impart softness and suppleness thereto. However, at least one of the cosmetic advantages mentioned above can unfortunately also be accompanied, on dried hair, by certain cosmetic effects considered undesirable, namely a lank effect on the hairstyle (lack of lightness of the hair) and a lack of smoothness (hair not uniform from the root to the end).

In addition, the use of aminosilicones for this purpose can have various drawbacks. On account of their strong affinity for hair, some of these silicones can become deposited in considerable amount during repeated use, and can lead to adverse effects such as an unpleasant, laden (charged or loaded) feel, stiffening of the hair and adhesion between fibres, affecting the styling. These drawbacks can be accentuated in the case of fine hair, which lacks liveliness and volume.

In summary, it is found that the current cosmetic compositions containing conditioners are not always entirely satisfactory.

The inventors have now discovered that the combination of at least one particular aminosilicone with at least one conditioner makes it possible to overcome at least one of these drawbacks.

The inventors have now found that a composition comprising at least one aminosilicone comprising at least one aminoethylimino(C₄-C₈)alkyl group and at least one conditioner, makes it possible to limit or even eliminate at least one of the lack of sheen, smoothness and softness of the hair, while at the same time retaining at least one of the other advantageous cosmetic properties associated with compositions containing a silicone.

The composition disclosed herein can also afford at least one improved cosmetic property (such as lightness, disentangling, volume, smoothness and sheen) and, what is more, the effects can be persistent and remanent.

The inventors have now found that the remanence of at least one cosmetic property (for example, the conditioning effects) can be improved by the presence of the at least one aminosilicone.

The compositions disclosed herein when applied to the skin, for example, in the form of a bubble bath or shower gel, can afford an improvement in the softness of the skin.

The compositions disclosed herein can also promote the deposition of conditioners onto keratin materials.

Thus, novel cosmetic compositions are now proposed, comprising, in a cosmetically acceptable medium, at least one aminosilicone comprising at least one aminoethylimino(C₄-C₈)alkyl group and at least one conditioner.

Another new embodiment relates to the inclusion of at least one aminosilicone comprising at least one aminoethylimino(C₄-C₈)alkyl group, in, or for the manufacture of, a cosmetic composition comprising at least one conditioner.

Another new embodiment relates to a composition comprising at least one aminosilicone comprising at least one aminoethylimino(C₄-C₈)alkyl group and at least one conditioner, as well as methods, for conditioning a keratin material.

Another new embodiment relates to a composition comprising at least one aminosilicone comprising at least one aminoethylimino(C₄-C₈)alkyl group and at least one conditioner, as well as methods, for improving lightness, softness, sheen and/or disentangling, and/or providing ease of styling of keratin materials.

Another new embodiment relates to a composition comprising at least one aminosilicone comprising at least one aminoethylimino(C₄-C₈)alkyl group and at least one conditioner, as well as methods, for improving persistent and/or remanent effects, for example, being able to withstand shampooing several times.

Another new embodiment relates to the at least one aminosilicone as defined below, as well as methods, for improving remanence of the conditioning effects of a cosmetic composition with respect to shampooing.

Another new embodiment relates to a process for improving remanence of the conditioning effects of a cosmetic composition with respect to shampooing comprising including the at least one aminosilicone as defined below in the cosmetic composition.

Various illustrative new embodiments will now be described in detail. All the meanings and definitions of the compounds given below are valid for all embodiments.

The term “keratin materials” means hair, eyelashes, eyebrows, skin, nails, mucous membranes or scalp.

In one new embodiment, the at least one aminosilicone is of formula (I) below:

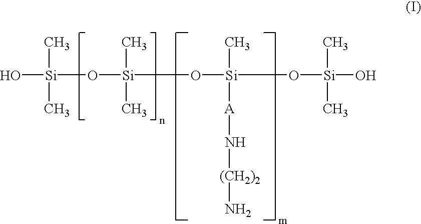

in which:

m and n are numbers such that the sum (n+m) ranges, for example, from 1 to 2000 and further, for example, from 50 to 150, n is a number ranging, for example, from 0 to 1999 and further, for example, from 49 to 149, and m is a number ranging, for example, from 1 to 2000 and further, for example, from 1 to 10;

A is chosen from linear and branched alkylene radicals comprising from 4 to 8 carbon atoms and, for example, 4 carbon atoms. In one embodiment, the alkylene radical can be, for example, linear.

The weight-average molecular mass of the at least one aminosilicone ranges, for example, from 2000 to 1 000 000 and further, for example, from 3500 to 200 000.

The weight-average molecular mass of the at least one aminosilicone is measured by gel permeation chromatography (GPC) at room temperature, in polystyrene equivalents. The columns used are styragel μ columns. The eluent is THF, and the flow rate is 1 ml/minute. 200 μl of a solution at 0.5% by weight of silicone in THF are injected. The detection is performed by refractometry and UV-metry.

The viscosity at 25° C. of the at least one aminosilicone is, for example, greater than 25 000 cSt (mm²/s) and further, for example, ranges from 30 000 to 200 000 cSt (mm²/s) and even further, for example, ranges from 30 000 to 150 000 cSt (mm²/s).

The at least one aminosilicone has, for example, an amine number of less than or equal to 0.4 meq/g, further, for example, ranging from 0.001 to 0.2 meq/g and even further, for example, ranging from 0.01 to 0.1 meq/g.

The amine number is the number of amine milliequivalents per gram of compound. This number is determined in an entirely conventional manner by means of titration methods using coloured indicator or by potentiometric titration.

In one new embodiment, the at least one aminosilicone is used in the form of an oil-in-water emulsion. The oil-in-water emulsion may comprise at least one surfactant.

The at least one surfactant may be of any nature, for example, cationic and/or nonionic.

The mean particle size of the at least one aminosilicone in the emulsion ranges, for example, from 3 to 500 nanometres, and further, for example, from 5 to 300 nanometres, and even further, for example, from 10 to 275 nanometres and even further, for example, from 150 to 275 nanometres. Such particle sizes are measured with a laser granulometer.

An example of the at least one aminosilicone corresponding to this formula is DC2-8299 from Dow Corning.

The at least one aminosilicone is, for example, used in an amount ranging from 0.01% to 20% by weight relative to the total weight of the composition. As a further example, this amount ranges from 0.1% to 15% by weight and even further, for example, ranges from 0.5% to 10% by weight relative to the total weight of the composition.

As used herein, the term “conditioner” means any agent whose function is to improve at least one cosmetic property of keratin materials such as hair, for example the softness, smoothness, disentangling, feel and static electricity.

The at least one conditioner may be soluble or insoluble in water.

The at least one conditioner is, for example, chosen from synthetic oils such as polyolefins, mineral oils, plant oils, fluoro oils and perfluoro oils, natural and synthetic waxes, compounds of ceramide type, carboxylic acid esters, silicones other than those comprising at least one aminoethylimino(C₄-C₈)alkyl group, anionic polymers, nonionic polymers, cationic polymers, amphoteric polymers, cationic proteins, cationic protein hydrolysates and cationic surfactants, usually used in cosmetic and/or dermatological compositions.

The insoluble conditioner may be solid, liquid or pasty at room temperature (25° C.) and at atmospheric pressure, and may, for example, be in the form of oils, waxes, resins or gums.

The insoluble conditioners can, for example, be dispersed in the compositions in the form of particles, which can have a number-average size ranging, for example, from 2 nanometres to 100 microns, and further, for example, from 30 nanometres to 20 microns. The number-average size of insoluble conditioner particles is measured with a laser granulometer.

The water-insoluble conditioners are insoluble in water at a concentration of greater than or equal to 0.1% by weight in water at 25° C., i.e. they do not form a macroscopically isotropic transparent solution under these conditions.

The synthetic oils are, for example, polyolefins, such as poly-α-olefins and, further, such as:

poly-α-olefins of hydrogenated and non-hydrogenated polybutene type, and even further such as hydrogenated and non-hydrogenated polyisobutenes.

Isobutylene oligomers with a molecular weight of less than 1000 and mixtures thereof with polyisobutylenes with a molecular weight of greater than 1000, such as from 1000 to 15 000, can, for example, be used.

Among the poly-α-olefins, which can be used, mention may be made, for example, of products sold under the name Permethyl® 99 A, 101 A, 102 A, 104 A (n=16) and 106 A (n=38) by the company Presperse Inc., products sold under the name Arlamol® HD (n=3) by the company ICI (n denoting the degree of polymerization), and

poly-α-olefins of hydrogenated and non-hydrogenated polydecene type.

Such products are sold, for example, under the names Ethylflo® by the company Ethyl Corp., and Arlamol® PAO by the company ICI.

The animal and plant oils are, for example, chosen from sunflower oil, corn oil, soybean oil, avocado oil, jojoba oil, marrow oil, grapeseed oil, sesame oil, hazelnut oil, fish oils, glyceryl tricaprocaprylate, and plant and animal oils of formula R₉COOR₁₀ in which R9 is chosen from higher fatty acid residues containing from 7 to 29 carbon atoms and R₁₀ is chosen from linear and branched hydrocarbon-based chains containing from 3 to 30 carbon atoms, such as alkyl and alkenyl, for example, purcellin oil and liquid jojoba wax.

It is also possible to use natural or synthetic essential oils such as eucalyptus oil, lavendin oil, lavender oil, vetiver oil, Litsea cubeba oil, lemon oil, sandalwood oil, rosemary oil, camomile oil, savory oil, nutmeg oil, cinnamon oil, hyssop oil, caraway oil, orange oil, geraniol oil, cade oil and bergamot oil.

The waxes are natural (animal or plant) or synthetic substances that are solid at room temperature (20°-25° C.). They are insoluble in water, soluble in oils and are capable of forming a water-repellent film.

Among the waxes, mention may be made, for example, of those discussed in P. D. Dorgan, Drug and Cosmetic Industry, December 1983, pp. 30-33.

The waxes are chosen, for example, from carnauba wax, candelilla wax, alfalfa wax, paraffin wax, ozokerite, plant waxes such as olive tree wax, rice wax, hydrogenated jojoba wax and absolute waxes of flowers such as the essential wax of blackcurrant flower sold by the company Bertin (France), animal waxes such as beeswaxes, and modified beeswaxes (cerabellina). Other waxes or waxy starting materials, which can be used, include, for example, marine waxes such as the product sold by the company Sophim under the reference M82, and polyethylene waxes and polyolefins in general.

The compounds of ceramide type are chosen, for example, from natural and synthetic ceramides, glycoceramides, pseudoceramides, and neoceramides.

Compounds of ceramide type are described, for example, in patent applications DE 4 424 530, DE 4 424 533, DE 4 402 929, DE 4 420 736, WO 95/23807, WO 94/07844, EP-A-0 646 572, WO 95/16665, FR-2 673 179, EP-A-0 227 994, WO 94/07844, WO 94/24097 and WO 94/10131, the teachings of which are incorporated herein by reference.

Compounds of ceramide type include, for example:

2-N-linoleoylaminooctadecane-1,3-diol,

2-N-oleoylaminooctadecane-1,3-diol,

2-N-palmitoylaminooctadecane-1,3-diol,

2-N-stearoylaminooctadecane-1,3-diol,

2-N-behenoylaminooctadecane-1,3-diol,

2-N-[2-hydroxypalmitoyl]aminooctadecane-1,3-dio,

2-N-stearoylaminooctadecane-1,3,4-triol such as N-stearoylphytosphingosine,

2-N-palmitoylaminohexadecane-1,3-diol,

bis(N-hydroxyethyl-N-cetyl)malonamide,

N-(2-hydroxyethyl)-N-(3-cetyloxy-2-hydroxypropyl)cetylamide,

N-docosanoyl-N-methyl-D-glucamine,

and mixtures of these compounds.

The fluoro oils are, for example, the perfluoropolyethers described, for example, in patent application EP-A-486 135 and the fluorohydrocarbon compounds described, for example, in patent application WO 93/11103. The teaching of these two patent applications is incorporated herein by reference.

The term “fluorohydrocarbon compounds” means compounds whose chemical structure contains a carbon skeleton in which at least one hydrogen atom has been replaced with a fluorine atom.

The fluoro oils can also be fluorocarbons such as fluoroamines, for example perfluorotributylamine, fluorohydrocarbons, for example perfluorodecahydronaphthalene, fluoro esters and fluoro ethers.

The perfluoropolyethers are sold, for example, under the trade names Fomblin by the company Montefluos and Krytox by the company Du Pont.

Among the fluorohydrocarbon compounds, mention may also be made, for example, of fluorine-containing fatty acid esters such as the product sold under the name Nofable FO by the company Nippon Oil.

The fatty alcohols may be chosen from linear and branched C₈-C₂₂ fatty alcohols, optionally oxyalkylenated with 1 to 15 mol of alkylene oxide or polyglycerolated with 1 to 6 mol of glycerol. The alkylene oxide is chosen, for example, from ethylene oxide and propylene oxide.

The carboxylic acid esters are chosen, for example, from mono-, di-, tri- and tetracarboxylic esters.

The monocarboxylic acid esters are chosen, for example, from linear and branched, saturated and unsaturated C₁-C₂₆ aliphatic acid monoesters of linear and branched, saturated and unsaturated, C₁-C₂₆ aliphatic alcohols, where the total carbon number of such esters being greater than or equal to 10.

Among the monoesters, mention may be made, for example, of dihydroabietyl behenate; octyidodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates, 2-ethylhexyl palmitate, 2-octyidecyl palmitate, alkyl myristates such as isopropyl, butyl, cetyl and 2-octyidodecyl myristate, hexyl stearate, butyl stearate, isobutyl stearate; dioctyl malate, hexyl laurate, and 2-hexyldecyl laurate.

C₄-C₂₂ di- or tricarboxylic acid esters of C₁-C₂₂ alcohols and mono-, di- or tricarboxylic acid esters of C₂-C₂₆ di-, tri-, tetra- or pentahydroxy alcohols can also be used.

Mention may be made, for example, of: diethyl sebacate; diisopropyl sebacate; diisopropyl adipate; di-n-propyl adipate; dioctyl adipate; diisostearyl adipate; dioctyl maleate; glyceryl undecylenate; octyldodecylstearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate dicaprate; tridecyl erucate; triisopropyl citrate; triisostearyl citrate; glyceryl trilactate; glyceryl trioctanoate; trioctyldodecyl citrate; and trioleyl citrate.

Among the esters mentioned above, mention may be made, for example, of ethyl and isopropyl palmitates, 2-ethylhexyl palmitate, 2-octyldecyl palmitate, alkyl myristates such as isopropyl, butyl, cetyl and 2-octyldodecyl myristate, hexyl stearate, butyl stearate, isobutyl stearate; dioctyl malate, hexyl laurate, 2-hexyldecyl laurate, isononyl isononanoate and cetyl octanoate.

The silicones other than those comprising at least one aminoethylimino(C₄-C₈)alkyl group, are chosen, for example, from polyorganosiloxanes that can be insoluble in the composition and may be in the form of oils, waxes, resins or gums.

The organopolysiloxanes are defined in greater detail in Walter Noll's “Chemistry and Technology of Silicones” (1968) Academic Press. They can be volatile or non-volatile.

The volatile organopolysiloxanes are, for example, chosen from those having a boiling point ranging from 60° C. to 260° C., and, further, for example, are chosen from:

(i) cyclic silicones comprising from 3 to 7 silicon atoms, such as 4 to 5 silicon atoms. These cyclic silicones are, for example, octamethylcyclotetrasiloxane sold, for example, under the name “Volatile Silicone 7207” by Union Carbide or “Silbione 70045 V 2” by Rhodia Chimie, decamethylcyclopentasiloxane sold under the name “Volatile Silicone 7158” by Union Carbide, and “Silbione 70045 V 5” by Rhodia Chimie, and mixtures thereof.

Mention may also be made, for example, of cyclocopolymers of the dimethylsiloxanes/methylalkylsiloxane type, such as “Volatile Silicone FZ 3109” sold by the company Union Carbide, with the chemical structure:

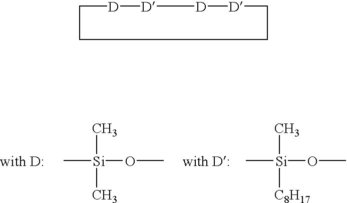

Mention may also be made, for example, of mixtures of cyclic silicones with organosilicone compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane; and (ii) linear volatile silicones comprising from 2 to 9 silicon atoms and having a viscosity of less than or equal to 5×10⁻⁶ m²/s at 25° C. An example is the decamethyltetrasiloxane sold, for example, under the name “SH 200” by the company Toray Silicone. Silicones belonging to this category are also described in the article published in Cosmetics and Toiletries, Vol. 91, Jan. 76, pp. 27-32, Todd & Byers “Volatile Silicone Fluids for Cosmetics”.

Non-volatile silicones, and, for example, polyalkylsiloxanes, polyarylsiloxanes, polyalkylarylsiloxanes, silicone gums and resins, polyorganosiloxanes modified with organofunctional groups, and mixtures thereof, may also be used.

These silicones are further, for example, chosen from polyalkylsiloxanes, among which mention may be made, for example, of polydimethylsiloxanes comprising trimethylsilyl end groups having a viscosity of from 5×10⁻⁶ to 2.5 m²/s at 25° C. and such as from 1×10⁻⁵ to 1 m²/s.

The viscosity of the silicones is measured at 25° C. according to ASTM standard 445 Appendix C.

Among these polyalkylsiloxanes, mention may be made, in a non-limiting manner, of the following commercial products:

the Silbione® oils of the 47 and 70 047 series or the Mirasil® oils sold by Rhodia Chimie, such as the oil 70 047 V 500 000;

the oils of the Mirasil series sold by the company Rhodia Chimie;

the oils of the 200 series from the company Dow Corning, such as DC200 with a viscosity of 60 000 cSt (mm²/s); and

the Viscasil® oils from General Electric and certain oils of the SF series (SF 96, SF 18) from General Electric.

Mention may also be made, for example, of polydimethylsiloxanes comprising dimethylsilanol end groups (Dimethiconol according to the CTFA name) such as the oils of the 48 series from the company Rhodia Chimie.

In this category of polyalkylsiloxanes, mention may also be made, for example, of the products sold under the names “Abil® Wax 9800 and 9801” by the company Goldschmidt, which are poly(C₁-C₂₀)alkylsiloxanes.

The polyalkylarylsiloxanes are chosen, for example, from linear and branched polydimethylmethylphenylsiloxanes and polydimethyldiphenylsiloxanes with a viscosity that ranges, for example, from 1×10⁵ to 5×10² m²/s at 25° C.

Among these polyalkylarylsiloxanes, mention may be made, for example, of the products sold under the following names:

the Silbione oils of the 70 641 series from Rhodia Chimie;

the oils of the Rhodorsil 70 633 and 763 series from Rhodia Chimie;

the oil Dow Corning 556 Cosmetic Grade Fluid from Dow Corning;

the silicones of the PK series from Bayer, such as the product PK20;

the silicones of the PN and PH series from Bayer, such as the products PN1000 and PH1000; and

certain oils of the SF series from General Electric, such as SF 1023, SF 1154, SF 1250 and SF 1265.

The silicone gums that can be used are, for example, chosen from polydiorganosiloxanes having the number-average molecular masses ranging from 200 000 to 1 000 000, used alone or as a mixture in at least one solvent. This solvent can, for example, be chosen from volatile silicones, polydimethylsiloxane (PDMS) oils, polyphenylmethylsiloxane (PPMS) oils, isoparaffins, polyisobutylenes, methylene chloride, pentane, dodecane and tridecanes, and mixtures thereof.

Mention may also be made, for example, of the following polymers:

polydimethylsiloxane

polydimethylsiloxane/methylvinylsiloxane gums,

polydimethylsiloxane/diphenylsiloxane,

polydimethylsiloxane/phenylmethylsiloxane, and

polydimethylsiloxane/diphenylsiloxane/methylvinylsiloxane.

Products, which can be used, for example are mixtures such as:

mixtures formed from a polydimethylsiloxane hydroxylated at the end of the chain (referred to as dimethiconol according to the nomenclature in the CTFA dictionary) and from a cyclic polydimethylsiloxane (referred to as cyclomethicone according to the nomenclature in the CTFA dictionary), such as the product Q2 1401 sold by the company Dow Corning;

mixtures formed from a polydimethylsiloxane gum with a cyclic silicone, such as the product SF 1214 Silicone Fluid from the company General Electric; this product is an SF 30 gum corresponding to a dimethicone, having a number-average molecular weight of 500 000, dissolved in the oil SF 1202 Silicone Fluid corresponding to decamethylcyclopentasiloxane; and

mixtures of two PDMSs of different viscosities, for example, of a PDMS gum and a PDMS oil, such as the product SF 1236 from the company General Electric. The product SF 1236 is a mixture of an SE 30 gum defined above, having a viscosity of 20 m²/s, and an SF 96 oil, with a viscosity of 5×10⁻⁶ m²/s. This product, for example, contains 15% SE 30 gum and 85% SF 96 oil.

The organopolysiloxane resins that can be used are crosslinked siloxane systems comprising at least one of the following units:

R₂SiO_{2/2}, R₃SiO_{1/2}, RSiO_{3/2} and SiO_{4/2} wherein R is chosen from hydrocarbon-based groups comprising from 1 to 16 carbon atoms and phenyl groups. Among these products, examples include the ones in which R is chosen from C₁-C₄ lower alkyl radicals, such as methyl, and a phenyl radical.

Among these resins, mention may be made, for example, of the product sold under the name “Dow Corning 593” or those sold under the names “Silicone Fluid SS 4230 and SS 4267” by the company General Electric, which are silicones of dimethyl/trimethyl siloxane structure.

Mention may also be made, for example, of the trimethyl siloxysilicate type resins sold, for example, under the names X22-4914, X21-5034 and X21-5037 by the company Shin-Etsu.

The organomodified silicones are silicones as defined above and comprising in their structure at least one organofunctional group attached via a hydrocarbon-based radical.

Among the organomodified silicones other than those comprising at least one aminoethylimino(C₄-C₈) group, mention may be made, for example, of polyorganosiloxanes comprising at least one group chosen from:

polyethylenoxy and polypropylenoxy groups optionally containing C₆-C₂₄ alkyl groups, such as the products known as dimethicone copolyol sold by the company Dow Corning under the name DC 1248 and the (C₁₋₂)alkylmethicone copolyol sold by the company Dow Corning under the name Q2 5200;

substituted and unsubstituted amine groups, such as the products sold under the name GP 4 Silicone Fluid and GP 7100 by the company Genesee, or the products sold under the names Q2 8220 and Dow Corning 929 or 939 by the company Dow Corning. The substituted amine groups are, for example, chosen from C₁-C₄ aminoalkyl groups;

thiol groups such as the products sold under the names “GP 72 A” and “GP 71” from Genesee;

alkoxylated groups such as the product sold under the name “Silicone Copolymer F-755” by SWS Silicones and Abil Wax 2428, 2434 and 2440 by the company Goldschmidt;

hydroxylated groups such as the polyorganosiloxanes comprising a hydroxyalkyl function, described in French patent application FR-A-85/16334;

acyloxyalkyl groups such as, for example, the polyorganosiloxanes described in patent U.S. Pat. No. 4,957,732;

anionic groups of carboxylic type, such as the products described in patent EP 186 507 from the company Chisso Corporation, and ionic groups of alkylcarboxylic type, such as those present in the product X-22-3701 E from the company Shin-Etsu; 2-hydroxyalkyl sulphonate; 2-hydroxyalkyl thiosulphate such as the products sold by the company Goldschmidt under the names “Abil S201” and “Abil S255”;

hydroxyacylamino groups, such as the polyorganosiloxanes described in patent application EP 342 834. Mention may be made, for example, of the product Q2-8413 from the company Dow Corning.

The silicones, which can also be used, comprise a polysiloxane portion and a portion comprising a non-silicone organic chain, one of the two portions constituting the main chain of the polymer, the other being grafted onto the main chain. Such polymers are described, for example, in patent applications EP-A-412 704, EP-A-412 707, EP-A-640 105, WO 95/00578, EP-A-582 152 and WO 93/23009 and U.S. Pat. Nos. 4,693,935, 4,728,571 and 4,972,037. These polymers are, for example, anionic or nonionic.

Such polymers are, for example, copolymers that can be obtained by radical polymerization starting with a monomer mixture comprising:

a) 50 to 90% by weight of tert-butyl acrylate;

b) 0 to 40% by weight of acrylic acid;

c) 5 to 40% by weight of silicone macromer of the formula (11):

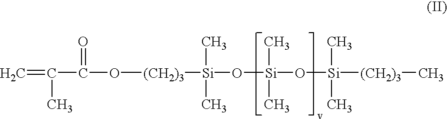

wherein v is a number ranging, for example, from 5 to 700; and the weight percentages is calculated relative to the total weight of the monomers.

Other examples of the grafted silicone polymers include, for example, polydimethylsiloxanes (PDMS) onto which are grafted, via a connecting chain unit of thiopropylene type, mixed polymer units of poly(meth)acrylic acid type and of polyalkyl (meth)acrylate type and polydimethylsiloxanes (PDMS) onto which are grafted, via a connecting chain unit of thiopropylene type, polymer units of polyisobutyl (meth)acrylate type.

All of the silicones can also be used in the form of emulsions, nanoemulsions or microemulsions.

The silicones, which can be used, include, for example:

non-volatile silicones chosen from polyalkylsiloxanes comprising trimethylsilyl end groups, such as oils having a viscosity ranging, for example, from 0.2 to 2.5 m²/s at 25° C., such as the oils of the DC200 series from Dow Corning, for example, oils with a viscosity of 60 000 cSt, of the Silbione 70047 and 47 series and, further, for example, the oil 70 047 V 500 000, which are sold by the company Rhodia Chimie, polyalkylsiloxanes comprising dimethylsilanol end groups, such as dimethiconols, and polyalkylarylsiloxanes such as the oil Silbione 70641 V 200 sold by the company Rhodia Chimie;

the polyorganosiloxane resin sold under the name Dow Corning 593; and

polysiloxanes comprising amine groups, such as amodimethicones other than those of formula (I) and trimethylsilylamodimethicones.

The conditioners that are mentioned below may be water-soluble, for example, anionic polymers, nonionic polymers, cationic polymers, amphoteric polymers, cationic proteins, cationic protein hydrolysates and cationic surfactants, and also mixtures of these various compounds.

The anionic polymers generally used are polymers comprising at least one group derived from carboxylic acid, sulphonic acid or phosphoric acid and such as those having a weight-average molecular weight ranging from 500 to 5 000 000, determined, for example, by gel permeation chromatography.

The anionic polymers may be, for example, chosen from:

1) polymers comprising carboxylic units derived from unsaturated mono- or dicarboxylic acid monomers such as those corresponding to the formula (III):

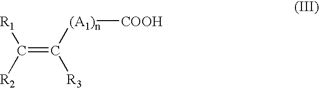

in which n is an integer ranging from 0 to 10, A₁ is a methylene group, optionally connected to the carbon atom of the unsaturated group, or to the neighbouring methylene group when n is greater than 1, via a hetero atom such as oxygen or sulphur, R₁ is chosen from a hydrogen atom and phenyl and benzyl groups, R₂ is chosen from a hydrogen atom and lower alkyl and carboxyl groups, R₃ is chosen from a hydrogen atom, lower alkyl groups and —CH₂—COOH, phenyl and benzyl groups;

In the above-mentioned formula, the lower alkyl radicals are chosen, for example, from alkyl radicals comprising 1 to 4 carbon atoms such as methyl and ethyl.

The anionic film-forming polymers comprising carboxylic groups may, for example, be chosen from:

A) acrylic and methacrylic acid homo- and copolymers, and salts thereof, such as the products sold under the names Versicol E or K by the company Ciba and Ultrahold by the company BASF. The copolymers of acrylic acid and of acrylamide sold in the form of their sodium salt under the names Reten 421, 423 or 425 by the company Hercules, the sodium salts of polyhydroxycarboxylic acids.

B) copolymers of acrylic and methacrylic acids with a monoethylenic monomer such as ethylene, styrene, vinyl esters, acrylic and methacrylic acid esters, optionally grafted onto a polyalkylene glycol such as polyethylene glycol and optionally crosslinked. Such polymers are described, for example, in French Patent No. 1,222,944 and German patent application 2,330,956, the copolymers of this type comprising an optionally N-alkylated and/or hydroxyalkylated acrylamide unit in their chain as described, for example in Luxembourg patent applications 75370 and 75371 or sold under the name Quadramer by the company American Cyanamid. Mention may also be made of copolymers of acrylic acid and of C₁-C₄ alkyl methacrylate and terpolymers of vinylpyrrolidone, of acrylic acid and of methacrylate of C₁-C₂₀ alkyl, for example lauryl, such as the product sold by the company ISP under the name Acrylidone LM and methacrylic acid/ethyl acrylate/tert-butyl acrylate terpolymers such as the product sold under the name Luvimer 100 P by the company BASF.

C) copolymers derived from crotonic acid such as those containing vinyl acetate or propionate units in their chain and, for example, other monomers such as allylic esters or methallylic esters, vinyl ether and vinyl ester of an acid chosen from linear and branched saturated carboxylic acids with a long hydrocarbon chain such as those containing at least carbon atoms, it being possible for these polymers optionally to be grafted and crosslinked, or vinyl, allylic and methallylic esters of an α- or β-cyclic carboxylic acid. Such polymers are described, inter alia, in French Patent Nos. 1 222 944, 1 580 545, 2 265 782, 2 265 781, 1 564 110 and 2 439 798. Commercial products falling into this class include the resins 28-29-30, 26-13-14 and 28-13-10 sold by the company National Starch.

D) copolymers derived from C₄-C₈ monounsaturated carboxylic acids or anhydrides chosen from:

copolymers comprising (i) at least one unit derived from maleic, fumaric and itaconic acids and anhydrides and (ii) at least one monomeric unit derived from vinyl esters, vinyl ethers, vinyl halides, phenylvinyl derivatives, acrylic acids and esters thereof, the anhydride functions of these copolymers optionally being monoesterified or monoamidated. Such polymers are described, for example, in U.S. Pat. Nos. 2,047,398, 2,723,248 and 2,102,113 and GB patent 839 805 and such as those sold under the names Gantrez AN or ES by the company ISP.

copolymers comprising (i) at least one unit chosen from maleic, citraconic and itaconic anhydrides and (ii) at least one unit chosen from monomeric units chosen from allylic and methallylic esters optionally containing at least one group chosen from acrylamide, methacrylamide, α-olefin, acrylic and methacrylic ester, acrylic and methacrylic acid and vinylpyrrolidone groups in their chain, the anhydride functions of these copolymers optionally being monoesterified or monoamidated.

These polymers are described, for example, in French patents 2 350 384 and 2 357 241.

E) polyacrylamides comprising carboxylate groups.

2) The polymers comprising sulphonic groups are, for example, polymers comprising vinylsulphonic, styrenesul phonic, naphthalenesulphonic or acrylamidoalkylsulphonic units.

These polymers can be chosen, for example, from:

polyvinylsulphonic acid salts having a weight-average molecular weight ranging, for example, from 1000 to 100 000, as well as the copolymers with an unsaturated comonomer such as acrylic or methacrylic acids and their esters, as well as acrylamide and its derivatives, vinyl ethers and vinylpyrrolidone;

polystyrenesulphonic acid salts, such as the sodium salts thereof having a weight-average molecular weight ranging, for example, from 500 000 to 100 000, which are sold under the names Flexan 500 and Flexan 130 by National Starch. These compounds are described in French Patent No. FR 2 198 719;

polyacrylamidesulphonic acid salts, those mentioned in U.S. Pat. No. 4,128,631 and, for example, polyacrylamidoethylpropanesulphonic acid sold under the name Cosmedia Polymer HSP 1180 by Cognis.

The anionic film-forming polymers may be, for example, chosen from acrylic acid copolymers, such as the acrylic acid/ethyl acrylate/N-tert-butylacrylamide terpolymer sold under the name Ultrahold Strong by the company BASF, copolymers derived from crotonic acid, such as the vinyl acetate/vinyl tert-butylbenzoate/crotonic acid terpolymers and the crotonic acid/vinyl acetate/vinyl neododecanoate terpolymers sold under the name Resin 28-29-30 by the company National Starch, polymers derived from maleic, fumaric or itaconic acids or anhydrides with vinyl esters, vinyl ethers, vinyl halides, phenylvinyl derivatives and acrylic acid and esters thereof, such as the methyl vinyl ether/monoesterified maleic anhydride copolymer sold under the name Gantrez ES 425 by the company ISP, the copolymers of methacrylic acid and methyl methacrylate sold under the name Eudragit L by the company Rohm Pharma, the copolymer of methacrylic acid and ethyl acrylate sold under the name Luvimer MAEX or MAE by the company BASF and the vinyl acetate/crotonic acid copolymer sold under the name Luviset CA 66 by the company BASF and the vinyl acetate/crotonic acid copolymer grafted with polyethylene glycol under the name Aristoflex A by the company BASF.

The anionic film-forming polymers may also be chosen, for example, from the methyl vinyl ether/monoesterified maleic anhydride copolymer sold under the name Gantrez ES 425 by the company ISP, the acrylic acid/ethyl acrylate/N-tert-butylacrylamide terpolymer sold under the name Ultrahold Strong by the company BASF, the copolymers of methacrylic acid and methyl methacrylate sold under the name Eudragit L by the company Rohm Pharma, the vinyl acetate/vinyl tert-butylbenzoate/crotonic acid terpolymers and the crotonic acid/vinyl acetate/vinyl neododecanoate terpolymers sold under the name Resin 28-29-30 by the company National Starch, the copolymer of methacrylic acid and ethyl acrylate sold under the name Luvimer MAEX or MAE by the company BASF and the vinylpyrrolidone/acrylic acid/lauryl methacrylate terpolymer sold under the name Acrylidone LM by the company ISP.

The amphoteric polymers that can be used can be chosen from polymers containing units K and M distributed randomly in the polymer chain, wherein K is a unit derived from a monomer comprising at least one basic nitrogen atom and M is a unit derived from an acidic monomer comprising at least one group chosen from carboxylic and sulphonic groups, or K and M can be chosen from groups derived from carboxybetaine or sulphobetaine zwitterionic monomers;

K and M can also be chosen from cationic polymer chains comprising at least one amine group chosen from primary, secondary, tertiary and quaternary amine groups, wherein at least one of the amine groups bears a carboxylic or sulphonic group connected via a hydrocarbon radical, or K and M can form part of a chain of a polymer comprising an α,β-dicarboxylic ethylene unit in which at least one of the carboxylic groups has been made to react with a polyamine comprising at least one amine group chosen from primary and secondary amine groups.

The amphoteric polymers corresponding to the definition given above, for example, are chosen from the following polymers:

(1) polymers resulting from the copolymerization of at least one monomer derived from a vinyl compound bearing a carboxylic group, such as acrylic acid, methacrylic acid, maleic acid, and α-chloroacrylic acid, and at least one basic monomer derived from a substituted vinyl compound comprising at least one basic atom, such as dialkylaminoalkyl methacrylates and acrylates, dialkylaminoalkylmethacrylamides and -acrylamides. Such compounds are described, for example, in U.S. Pat. No. 3,836,537. Mention may also be made, for example, of the sodium acrylate/acrylamidopropyltrimethylammonium chloride copolymer sold under the name “Polyquart KE 3033” by the company Cognis.

The vinyl compound may also be a dialkyldiallylammonium salt such as dimethyldiallylammonium chloride. The copolymers of acrylic acid and of the latter monomer are sold under the names “Merquat 280”, “Merquat 295” and “Merquat Plus 3330” by the company Nalco.

(2) polymers comprising units derived from:

a) at least one monomer chosen from acrylamides and methacrylamides substituted on the nitrogen with an alkyl radical,

b) at least one acidic comonomer comprising at least one reactive carboxylic group, and

c) at least one basic comonomer such as esters comprising substituents chosen from primary, secondary, tertiary and quaternary amine substituents of acrylic and methacrylic acids and the product of quaternization of dimethylaminoethyl methacrylate with dimethyl or diethyl sulphate.

In one embodiment, the N-substituted acrylamides or methacrylamides are, for example, groups in which the alkyl radicals comprise from 2 to 12 carbon atoms, such as N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers are chosen, for example, from acrylic acids, methacrylic acids, crotonic acids, itaconic acids, maleic acids and fumaric acids and alkyl monoesters, comprising from 1 to 4 carbon atoms, of maleic or fumaric acids or anhydrides.

The basic comonomers are chosen, for example, from aminoethyl, butylaminoethyl, N,N′-dimethylaminoethyl and N-tert-butylaminoethyl methacrylates.

The copolymers having the CTFA (4th edition, 1991) name octylacrylamide/acrylates/butylaminoethyl methacrylate copolymer such as the products sold under the name Amphomer or Lovocryl 47 by the company National Starch may, for example, also be used.

(3) crosslinked and partially or totally alkylated polyamino amides derived from polyamino amides of general formula (IV):

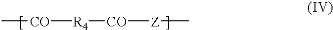

wherein R₄ is chosen from a divalent radical derived from a saturated dicarboxylic acid, mono- and dicarboxylic aliphatic acids comprising an ethylenic double bond, an ester of a lower alkanol, comprising from 1 to 6 carbon atoms, of these acids and a radical derived from the addition of any one of the acids to amines chosen from bis(primary) and bis(secondary) amines, and Z is chosen from bis(primary), mono- and bis(secondary) polyalkylene-polyamine radicals and, for example, represents:

a) in proportions ranging from 60 to 100 mol %, the radical of formula (V):

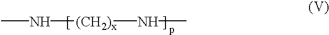

wherein x=2 and p=2 or 3, or x=3 and p=2

this radical being derived from a compound chosen from diethylenetriamine, triethylenetetraamine and dipropylenetriamine;

b) in proportions ranging from 0 to 40 mol %, the radical (V) above in which x=2 and p=1 and which is derived a compound chosen from ethylenediamine and piperazine:

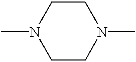

c) in proportions ranging from 0 to 20 mol %, the —NH—(CH₂)₆—NH— radical, which is derived from hexamethylenediamine, these polyaminoamines can be crosslinked by addition of a difunctional crosslinking agent chosen from epihalohydrins, diepoxides, dianhydrides and bis-unsaturated derivatives, using from 0.025 to 0.35 mol of crosslinking agent per amine group of the polyamino amide and alkylated by the action of acrylic acid, chloroacetic acid or an alkane sultone, or salts thereof.

In one embodiment, the saturated carboxylic acids are, for example, chosen from acids comprising from 6 to 10 carbon atoms, such as adipic acid, 2,2,4-trimethyladipic acid and 2,4,4-trimethyladipic acid, terephthalic acid, acids comprising an ethylenic double bond, such as acrylic acid, methacrylic acid and itaconic acid.

The alkane sultones used in the alkylation are chosen, for example, from propane sultone and butane sultone, and the salts of the alkylating agents may be chosen, for example, from the sodium and potassium salts.

(4) polymers comprising zwitterionic units of formula (VI):

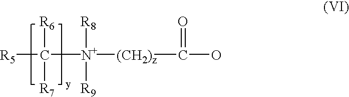

wherein R₅ is chosen from polymerizable unsaturated groups such as acrylate, methacrylate, acrylamide and methacrylamide groups, y and z, which may be identical or different, are chosen from integers ranging from 1 to 3, R₆ and R₇, which may be identical or different, are chosen from a hydrogen atom, and methyl, ethyl and propyl groups, R₈ and R₉, which may be identical or different, are chosen from a hydrogen atom and alkyl radicals such that the sum of the carbon atoms in R₈ and R₉ does not exceed 10.

The polymers comprising such units can also comprise units derived from non-zwitterionic monomers such as monomers chosen from dimethyl and diethylaminoethyl acrylates, methacrylates, alkyl acrylates, methacrylates, acrylamides, methacrylamides, and vinyl acetate.

By way of example, mention may be made of the copolymer of methyl methacrylate/dimethyl carboxymethylammonio methyl ethylmethacrylate such as the product sold under the name “Diaformer Z301” by the company Sandoz.

(5) polymers derived from chitosan comprising monomer units corresponding to formulae

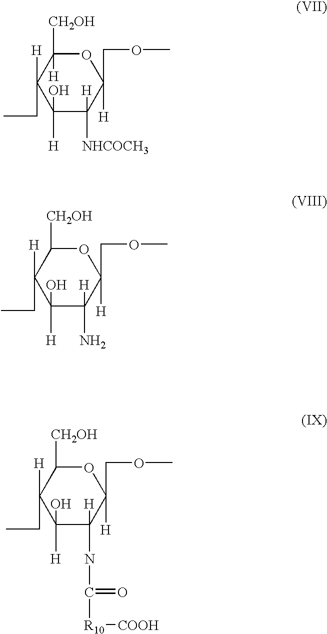

the unit (VII) being present in proportions ranging from 0 to 30%, the unit (VIII) in proportions ranging from 5 to 50% and the unit (IX) in proportions ranging from 30 to 90%, wherein in the unit (IX), R₁₀ is a radical of formula:

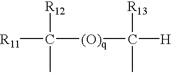

wherein q is chosen from 0 and 1;

if q=0, R₁₁, R₁₂ and R₁₃, which may be identical or different, are each chosen from a hydrogen atom, methyl, hydroxyl, acetoxy and amino residues, and monoalkylamine residues and dialkylamine residues which are optionally interrupted by at least one nitrogen atom and/or optionally substituted with at least one group chosen from amine, hydroxyl, carboxyl, alkylthio and sulphonic groups, and alkylthio residues in which the alkyl group bears an amino residue, at least one of the radicals R₁₁, R₁₂ and R₁₃ being, in this case, a hydrogen atom;

or, if q=1, R₁₁, R₁₂ and R₁₃, which may be identical or different, are each chosen from a hydrogen atom, and salts formed by these compounds with bases or acids.

(6) Polymers derived from the N-carboxyalkylation of chitosan, such as N-carboxymethylchitosan and N-carboxybutylchitosan sold under the name “Evalsan” by the company Jan Dekker.

(7) Polymers corresponding to the general formula (X) such as those described, for example, in French Patent No. 1 400 366:

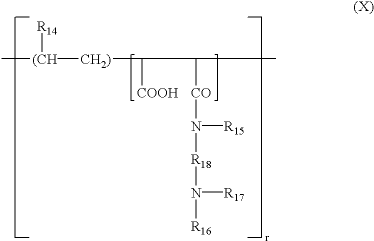

wherein R₁₄ is chosen from a hydrogen atom, and CH₃O, CH₃CH₂O and phenyl radicals, R₁₅ is chosen from a hydrogen atom and lower alkyl radicals such as methyl and ethyl, R₁₆ is chosen from a hydrogen atom and lower alkyl radicals such as methyl and ethyl, R₁₇ is chosen from lower alkyl radicals such as methyl and ethyl and radicals corresponding to the formula: —R₁₈—N(R₁₆)₂, wherein R₁₈ is chosen from —CH₂—CH₂—, —CH₂—CH₂—CH₂— and —CH₂—CH(CH₃)— groups, and R₁₆ is chosen from a hydrogen atom and lower alkyl radicals,

such as methyl and ethyl,

and the higher homologues of these radicals comprising up to 6 carbon atoms r is chosen such that the number-average molecular weight of said polymer ranges from 500 to 6 000 000, such as from 1000 to 1 000 000.

(8) Amphoteric polymers of the type -D-X-D-X chosen from:

a) polymers obtained by the action of chloroacetic acid or sodium chloroacetate on compounds comprising at least one unit of formula:

-D-X-D-X-D- (XI)

wherein D is a radical

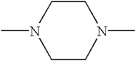

and X is chosen from the symbols E and E′, wherein E and E′, which may be identical or different, are chosen from divalent alkylene radicals comprising at least one chain chosen from straight and branched chains comprising up to 7 carbon atoms in the main chain, wherein the divalent alkylene radicals are unsubstituted or substituted with at least one hydroxyl group. E or E′ can additionally comprise at least one atom chosen from oxygen, nitrogen and sulphur atoms, and 1 to 3 rings chosen from aromatic and heterocyclic rings. The oxygen, nitrogen and sulphur atoms can be present in the form of at least one group chosen from ether, thioether, sulphoxide, sulphone, sulphonium, alkylamine and alkenylamine, hydroxyl, benzylamine, amine oxide, quaternary ammonium, amide, imide, alcohol, ester and urethane groups.

b) Polymers of formula:

-D-X-D-X- (XII)

wherein D is a radical

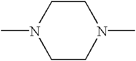

and X is chosen from the symbol E and E′ and wherein at least one X is chosen from E′; E having the meaning given above and E′ is a divalent radical chosen from divalent alkylene radicals comprising at least one chain chosen from straight and branched chains comprising up to 7 carbon atoms in the main chain, wherein said divalent alkylene radicals are optionally substituted with at least one hydroxyl radical and comprise at least one nitrogen atom substituted with an alkyl chain, which is optionally interrupted by an oxygen atom, and further comprising at least one functional group chosen from carboxyl functional groups and hydroxyl functional groups which are betainized by reaction with a reactant chosen from chloroacetic acid and sodium chloroacetate.

(9) (C₁-C₅)alkyl vinyl ether/maleic anhydride copolymers, the maleic anhydride being partially modified by semiamidation with an N,N-dialkylaminoalkylamine such as N,N-dimethylaminopropylamine or by semiesterification with an N,N-dialkanolamine. These copolymers can also comprise other vinyl comonomers such as vinylcaprolactam.

In one embodiment, the amphoteric polymers are chosen from polymers of family (1).

The nonionic polymers that can be used are chosen, for example, from the following polymers:

vinylpyrrolidone homopolymers;

copolymers of vinylpyrrolidone and vinyl acetate;

polyalkyloxazolines such as the polyethyloxazolines sold by the company Dow Chemical under the names “Peox 50 000”, “Peox 200 000” and “Peox 500 000”;

vinyl acetate homopolymers, such as the product sold under the name “Appretan EM” by the company Hoechst, and the product sold under the name “Rhodopas A 012” by the company Rhodia Chimie;

copolymers of vinyl acetate and acrylic ester, such as the product sold under the name “Rhodopas AD 310” by Rhodia Chimie;

copolymers of vinyl acetate and ethylene, such as the product sold under the name “Appretan TV” by the company Hoechst;

copolymers of vinyl acetate and maleic ester, for example of dibutyl maleate, such as the product sold under the name “Appretan MB Extra” by the company Hoechst;

copolymers of polyethylene and maleic anhydride;

alkyl acrylate homopolymers and alkyl methacrylate homopolymers, such as the product sold under the name “Micropearl RQ 750” by the company Matsumoto or the product sold under the name “Luhydran A 848 S” by the company BASF;

acrylic ester copolymers such as copolymers of alkyl acrylates and alkyl methacrylates, such as the products sold by the company Rohm & Haas under the names “Primal AC-261 K” and “Eudragit NE 30 D”, by the company BASF under the names “Acronal 601”, “Luhydran LR 8833” and 8845, and by the company Hoechst under the names “Appretan N 9213” or N 9212;

copolymers of acrylonitrile and a nonionic monomer chosen, for example, from butadiene and alkyl (meth)acrylates; mention may be made of the products sold under the names “Nipol LX 531 B” by the company Nippon Zeon and those sold under the name “CJ 0601 B” by the company Rohm & Haas;

polyurethanes, such as the products sold under the names “Acrysol RM 1020” and “Acrysol RM 2020” by the company Rohm & Haas, and the products “Uraflex XP 401 UZ” and “Uraflex XP 402 UZ” by the company DSM Resins;

copolymers of alkyl acrylate and urethane, such as the product “8538-33” by the company National Starch;

polyamides, such as the product “Estapor LO 11” sold by the company Rhodia Chimie; and

unmodified and chemically modified nonionic guar gums.

The unmodified nonionic guar gums are, for example, the products sold under the name “Vidogum GH 175” by the company Unipectine and under the name “Jaguar C” by the company Meyhall.

The modified nonionic guar gums that can be used are, for example, modified with at least one group chosen from C₁-C₆ hydroxyalkyl groups. Mention may be made, for example, of hydroxymethyl, hydroxyethyl, hydroxypropyl and hydroxybutyl groups.

These guar gums are well known in the state of the art and can be prepared, for example, by reacting corresponding alkene oxides, such as propylene oxides, with guar gum so as to obtain a guar gum modified with hydroxypropyl groups.

Such nonionic guar gums optionally modified with hydroxyalkyl groups are sold, for example, under the trade names “Jaguar HP8”, “Jaguar HP60” and “Jaguar HP120”, “Jaguar DC 293” and “Jaguar HP 105” by the company Meyhall and under the name “Galactasol 4H₄FD2” by the company Aqualon.

The alkyl radicals of the nonionic polymers comprise from 1 to 6 carbon atoms, except where otherwise mentioned.

Functionalized and non-functionalized polyurethanes may also be used as polymers.

The polyurethanes that may be used are, for example, those described in the documents of EP 0 751 162, EP 0 637 600, FR 2 743 297 and EP 0 648 485, and also the documents of EP 0 656 021 and WO 94/03510 and the document of EP 0 619 111.

The conditioners of cationic polymer type that may be used may be chosen from any of those already known per se by those skilled in the art as improving at least one cosmetic property of hair treated with detergent compositions, such as those described in patent application EP-A-0 337 354 and in French patent applications FR-A-2 270 846, 2 383 660, 2 598 611, 2 470 596 and 2 519 863.

Even more generally, the expression “cationic polymer” denotes any polymer containing cationic groups and/or groups that may be ionized into cationic groups.

The cationic polymers are, for example, chosen from those comprising units comprising at least one amine group chosen from primary, secondary, tertiary and quaternary amine groups that may either form part of the main polymer chain, or may be borne by a side substituent that is directly attached to the main polymer chain.

The cationic polymers generallyhave a number-average molar mass ranging, for example, from 500 to 5×10⁶ and such as, from 10³ to 3×10⁶.

Among the cationic polymers, mention may be made, for example, of polymers of the polyamine, polyamino amide and polyquaternary ammonium type. These polymers are known in the art.

The polymers of polyamine, polymers of polyamino amide and polymers of polyquaternary ammonium type that may be used, for example, are those described in French Patent Nos. 2 505 348 and 2 542 997. Among these polymers, mention may be made of:

(1) homopolymers and copolymers derived from acrylic or methacrylic esters or amides and comprising at least one of the units of the following formulae:

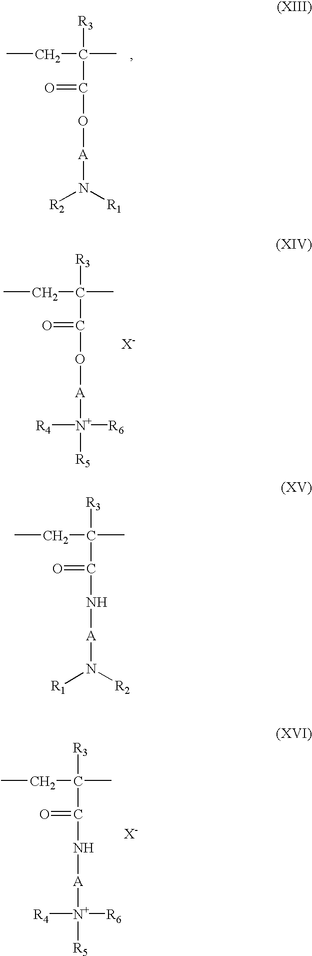

wherein:

R₁ and R₂, which may be identical or different, are chosen from a hydrogen atom and alkyl groups comprising from 1 to 6 carbon atoms, such as methyl and ethyl groups;

R₃, which may be identical or different, is chosen from a hydrogen atom and a CH₃ radical;

A, which may be identical or different, is chosen from linear and branched alkyl groups of 1 to 6 carbon atoms, such as 2 or 3 carbon atoms, or hydroxyalkyl groups of 1 to 4 carbon atoms;

R₄, R₅ and R₆, which may be identical or different, are chosen from alkyl groups comprising from 1 to 18 carbon atoms and benzyl radicals such as alkyl groups comprising from 1 to 6 carbon atoms;

X⁻ is an anion derived from a mineral or organic acid, such as a methosulphate anion or an anion chosen from halides such as chloride and bromide.

Copolymers of family (1) can also comprise at least one unit derived from comonomers which may be chosen from the family of acrylamides, methacrylamides, diacetone acrylamides, acrylamides and methacrylamides substituted on the nitrogen with at least one group chosen from lower (C₁-C₄) alkyls, acrylic acids, methacrylic acids, acrylic esters, methacrylic esters, vinyllactams such as vinylpyrrolidone and vinylcaprolactam, and vinyl esters.

Thus, among these copolymers of family (1), mention may be made of:

the copolymers of acrylamide and of dimethylaminoethyl methacrylate quaternized with dimethyl sulphate or with a dimethyl halide, such as the product sold under the name “Hercofloc” by the company Hercules,

the copolymers of acrylamide and of methacryloyloxyethyltrimethylammonium chloride described, for example, in patent application EP-A-080 976 and sold under the name “Bina Quat P 100” by the company Ciba,

the copolymer of acrylamide and of methacryloyloxyethyltrimethylammonium methosulphate sold under the name “Reten” by the company Hercules,

quaternized or non-quaternized vinylpyrrolidone/dialkylaminoalkyl acrylate or methacrylate copolymers, such as the products sold under the name “Gafquat” by the company ISP, for example, “Gafquat® 734” or “Gafquat® 755”, or the products known as “Copolymer 845, 958 and 937”. These polymers are described in detail in French Patent Nos. 2 077 143 and 2 393 573,

dimethylaminoethyl methacrylate/vinylcaprolactam/vinylpyrrolidone terpolymers, such as the product sold under the name “Gaffix® VC 713” by the company ISP,

vinylpyrrolidone/methacrylamidopropyldimethylamine copolymers sold, for example, under the name “Styleze® CC 10” by ISP, and

quaternized vinylpyrrolidone/dimethylaminopropylmethacrylamide copolymers such as the product sold under the name “Gafquat® HS 100” by the company ISP.

(2)cationic polysaccharides, for example, cationic celluloses and cationic galactomannan gums. Among the cationic polysaccharides that may be mentioned, for example, are cellulose ether derivatives comprising quaternary ammonium groups, cationic cellulose copolymers or cellulose derivatives grafted with a water-soluble quaternary ammonium monomer and cationic galactomannan gums.

The cellulose ether derivatives comprising quaternary ammonium groups are described in French Patent No. 1 492 597, and, for example, the polymers sold under the names “JR” (JR 400, JR 125, JR 30M) or “LR” (LR 400, LR 30M) by the company Amerchol. These polymers are also defined in the CTFA dictionary as hydroxyethylcellulose quaternary ammoniums that have reacted with an epoxide substituted with a trimethylammonium group.

The cationic cellulose copolymers or cellulose derivatives grafted with a water-soluble quaternary ammonium monomer are described, for example, in U.S. Pat. No. 4,131,576, such as hydroxyalkylcelluloses, for instance hydroxymethylcelluloses, hydroxyethylcelluloses and hydroxypropylcelluloses grafted, for example, with a salt chosen from methacryloylethyltrimethylammonium salts, methacrylamidopropyltrimethylammonium salts, and dimethyldiallylammonium salts.

The commercial products corresponding to this definition are, for example, the products sold under the name “Celquat® L 200” and “Celquat® H 100” by the company National Starch.

The cationic galactomannan gums are described, for example, in U.S. Pat. Nos. 3,589,578 and 4,031,307, such as guar gums comprising trialkylammonium cationic groups. For example, guar gums modified with a salt (e.g. chloride) of 2,3-epoxypropyltrimethylammonium may be used.

Such polymers are sold, for example, under the trade names “Jaguar® C13S”, “Jaguar® C15”, “Jaguar® C17” and “Jaguar® C162” by the company Meyhall.

(3) polymers comprising piperazinyl units and divalent alkylene or hydroxyalkylene radicals comprising straight or branched chains, optionally interrupted by at least one atom chosen from oxygen, sulphur and nitrogen atoms or by at least one ring chosen from aromatic and heterocyclic rings, and also at least one of the oxidation and quaternization products of these polymers. Such polymers are described, for example, in French Patent Nos. 2 162 025 and 2 280 361;

(4) water-soluble polyamino amides prepared, for example, by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or with an oligomer resulting from the reaction of a difunctional compound, which is reactive with a bis-halohydrin, a bis-azetidinium, a bis-haloacyldiamine, a bis-alkyl halide, an epihalohydrin, a diepoxide or a bis-unsaturated derivative. The crosslinking agent can be used in proportions ranging from 0.025 to 0.35 mol per amine group of the polyamino amide. These polyamino amides can be alkylated or, if they comprise at least one tertiary amine function, they can be quaternized. Such polymers are described, for example, in French Patent Nos. 2 252 840 and 2 368 508;

(5) polyamino amide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers in which the alkyl radical comprises from 1 to 4 carbon atoms and, for example, methyl, ethyl and propyl. Such polymers are described, for example, in French Patent No. 1 583 363.

Among these derivatives, mention may be made, for example, of the adipic acid/dimethylaminohydroxypropyl/diethylenetriamine polymers sold under the name “Cartaretine® F, F4 or F8” by the company Sandoz.

(6) polymers obtained by reaction of a polyalkylene polyamine comprising two primary amine groups and at least one secondary amine group with a dicarboxylic acid chosen from diglycolic acid and saturated aliphatic dicarboxylic acids comprising from 3 to 8 carbon atoms. The molar ratio between the polyalkylene polyamine and the dicarboxylic acid may range, for example, from 0.8:1 to 1.4:1; the polyamino amide resulting therefrom may be reacted with epichlorohydrin in a molar ratio of epichlorohydrin relative to the secondary amine group of the polyamino amide ranging from 0.5:1 to 1.8:1. Such polymers are described, for example, in U.S. Pat. Nos. 3,227,615 and 2,961,347.

Other non-limiting examples of such derivatives include the adipic acid/epoxypropyl/diethylenetriamine copolymers sold, for example, under the name “Hercosett® 57” by the company Hercules Inc. or under the name “PD 170” or “Delsette® 101” by the company Hercules.

(7) cyclopolymers of alkyldiallylamine or of dialkyldiallylammonium, such as the homopolymers or copolymers comprising, as main constituent of the chain, at least one unit corresponding to formula (XVII) or (XVIII):

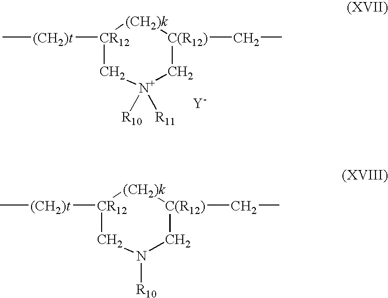

wherein k and t are equal to 0 or 1, the sum k+t being equal to 1; R₁₂ is chosen from a hydrogen atom and a methyl radical; R₁₀ and R₁₁, which may be identical or different, are chosen from alkyl groups comprising from 1 to 8 carbon atoms, hydroxyalkyl groups in which the alkyl group, for example, comprises 1 to 5 carbon atoms, and lower (C₁-C₄) amidoalkyl groups, or R₁₀ and R₁₁ are chosen from, together with the nitrogen atom to which they are attached, heterocyclic groups such as piperidyl or morpholinyl; Y⁻ is an anion such as bromide, chloride, acetate, borate, citrate, tartrate, bisulphate, bisulphite, sulphate and phosphate. These polymers are described, for example, in French Patent No. 2 080 759 and in its Certificate of Addition 2 190 406.

In one embodiment, R₁₀ and R₁₁, which may be identical or different, are, for example, chosen from alkyl groups comprising from 1 to 4 carbon atoms.

Among the polymers defined above, mention may be made, for example, of the dimethyldiallylammonium chloride homopolymer sold under the name “Merquat® 100” by the company Nalco (and its homologues of low weight-average molecular mass) and copolymers of diallyldimethylammonium chloride and of acrylamide, sold under the name “Merquat® 550”.

(8) quaternary diammonium polymers comprising repeating units corresponding to the formula (XIX):

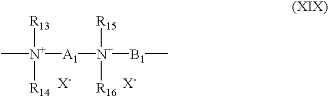

wherein:

R₁₃, R₁₄, R₁₅ and R₁₆, which may be identical or different, are chosen from aliphatic, alicyclic and arylaliphatic radicals comprising from 1 to 20 carbon atoms and from lower hydroxyalkylaliphatic radicals, or R₁₃, R₁₄, R₁₅ and R₁₆, together or separately, constitute, with the nitrogen atoms to which they are attached, heterocycles optionally comprising a second hetero atom other than nitrogen, or R₁₃, R₁₄, R₁₅ and R₁₆ are chosen from linear and branched C₁-C₆ alkyl radicals substituted with at least one group chosen from nitrile, ester, acyl and amide groups and groups of formulae —CO—O—R₁₇-D and —CO—NH—R₁₇-D wherein R₁₇ is chosen from alkylene groups and D is chosen from quaternary ammonium groups;

A₁ and B₁, which may be identical or different, are chosen from linear and branched, saturated and unsaturated polymethylene groups comprising from 2 to 20 carbon atoms. The polymethylene groups may comprise, linked to or intercalated in the main chain, at least one entity chosen from aromatic rings, oxygen, and sulphur atoms and sulphoxide, sulphone, disulphide, amino, alkylamino, hydroxyl, quaternary ammonium, ureido, amide and ester groups, and

X⁻ is an anion chosen from anions derived from mineral and organic acids;

A₁, R₁₃ and R₁₅ may optionally form, with the two nitrogen atoms to which they are attached, a piperazine ring. In addition, if A₁ is a radical chosen from linear and branched, saturated and unsaturated alkylene and hydroxyalkylene radicals, B₁ can also represent a group (CH₂)ₙ—CO-D-OC—(CH₂)ₙ—, wherein n ranges from 1 to 100, such as from 1 to 50, D is chosen from:

a) a glycol residue of formula: —O-Z-O—, wherein Z is chosen from linear and branched hydrocarbon-based radicals and a group corresponding to one of the following formulae:

—(CH₂—CH₂—O)ₓ—CH₂—CH₂—

—[CH₂—CH(CH₃)—O]_{y}—CH₂—CH(CH₃)—

wherein x and y, which may be identical or different, are each an integer ranging from 1 to 4, representing a defined and unique degree of polymerization or any number ranging from 1 to 4 representing an average degree of polymerization;

b) a bis-secondary diamine residue such as a piperazine derivative;

c) a bis-primary diamine residue of formula: —NH—Y—NH—, wherein Y is chosen from linear and branched hydrocarbon-based radicals, and the divalent radical —CH₂—CH₂—S—S—CH₂—CH₂—; and

d) a ureylene group of formula: —NH—CO—NH—.

In one embodiment, X— is an anion such as chloride or bromide.

These polymers may have a number-average molecular mass ranging from 1000 to 100 000.

These polymers are described, for example, in French Patent Nos. 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

Further, polymers can comprise repeating units corresponding to the formula (XX):

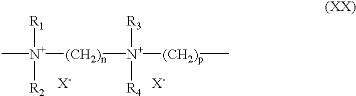

in which R₁, R₂, R₃ and R₄, which may be identical or different, are chosen from alkyl and hydroxyalkyl radicals comprising from 1 to 4 carbon atoms, n and p, which may be identical or different, are integers ranging from 2 to 20, and X— is an anion chosen from anions derived from mineral acids and organic acids.

One compound of formula (XX), for example, is the one for which R₁, R₂, R₃ and R₄ are each a methyl radical and n=3, p=6 and X=Cl, which is known as Hexadimethrine chloride according to the INCI (CTFA) nomenclature.

(9) polyquaternary ammonium polymers comprising repeating units of formula (XXI):

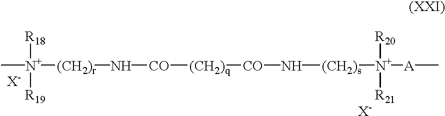

wherein:

R₁₈, R₁₉, R₂₀ and R₂₁, which may be identical or different, are chosen from a hydrogen atom and methyl, ethyl, propyl, β-hydroxyethyl, β-hydroxypropyl and —CH₂CH₂(OCH₂CH₂)ₚOH radicals,

wherein p is equal to 0 or to an integer ranging from 1 to 6, with the proviso that R₁₈, R₁₉, R₂₀ and R₂₁ do not simultaneously represent a hydrogen atom,

r and s, which may be identical or different, are each an integer ranging from 1 to 6,

q is equal to 0 or to an integer ranging from 1 to 34,

X⁻ is an anion such as a halide,

A is chosen from divalent radicals such as —CH₂—CH₂—O—CH₂—CH₂—.

Such polymers are described, for example, in patent application EP-A-122 324.

Among these polymers, mention may be made, for example, of “Mirapol® A 15”, “Mirapol® AD1”, “Mirapol® AZ1” and “Mirapol® 175” sold by the company Miranol.

(10) quaternary polymers of vinylpyrrolidone and of vinylimidazole, such as, for example, the products sold under the names “Luviquat® FC 905, FC 550 and FC 370” by the company BASF.

(11) polyamines such as the product “Polyquart® H” sold by Cognis under the reference name “Polyethylene Glycol (15) Tallow Polyamine” in the CTFA dictionary.

(12) crosslinked methacryloyloxy(C₁-C₄)alkyltri(C₁-C₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound comprising olefinic unsaturation, for example, methylenebisacrylamide. In one embodiment, a crosslinked acrylamide/methacryloyloxyethyltrimethylammoniu m chloride copolymer (20/80 by weight) in the form of a dispersion comprising 50% by weight of the said copolymer in mineral oil may be used. This dispersion is sold under the name “Salcare® SC 92” by the company Ciba. In another embodiment, a crosslinked homopolymer of methacryloyloxyethyltrimethylammonium chloride comprising 50% by weight of the homopolymer in mineral oil or in a liquid ester may also be used. These dispersions are sold under the names “Salcare® SC 95” and “Salcare® SC 96” by the company Ciba.

Other cationic polymers, which can be used, are chosen from cationic proteins and cationic protein hydrolysates, polyalkyleneimines, such as polyethyleneimines, polymers comprising unites chosen from vinylpyridine and vinylpyridinium units, condensates of polyamines and of epichlorohydrin, quaternary polyureylenes and chitin derivatives.

Among all the cationic polymers that may be used, non-limiting examples include quaternary cellulose ether derivatives such as the products sold under the name “JR 400” by the company Amerchol, cationic cyclopolymers, such as the dimethyldiallylammonium chloride homopolymers and copolymers sold under the names “Merquat® 100”, “Merquat® 550” and “Merquat® S” by the company Nalco, quaternary polymers of vinylpyrrolidone and of vinylimidazole, and mixtures thereof.

The cationic proteins or protein hydrolysates can be, for example, chemically modified polypeptides comprising quaternary ammonium groups at the end of the chain or grafted thereto. Their molecular mass can range, for example, from 1500 to 10 000 and further, for example, from 2000 to 5000. Among these compounds, mention may be made, for example, of:

collagen hydrolysates comprising triethylammonium groups, such as the products sold under the name “Quat-Pro E” by the company Maybrook and referred to in the CTFA dictionary as “Triethonium Hydrolyzed Collagen Ethosulphate”;

collagen hydrolysates comprising trimethylammonium and trimethylstearylammonium chloride groups, sold under the name “Quat-Pro S” by the company Maybrook and referred to in the CTFA dictionary as “Steartrimonium Hydrolyzed Collagen”;

animal protein hydrolysates comprising trimethylbenzylammonium groups such as the products sold under the name “Crotein® BTA” by the company Croda and referred to in the CTFA dictionary as “Benzyltrimonium hydrolyzed animal protein”; and

protein hydrolysates bearing, on the polypeptide chain, quaternary ammonium groups containing at least one alkyl radical comprising from 1 to 18 carbon atoms.

Among these protein hydrolysates, mention may be made of, inter alia:

“Croquat® L” in which the quaternary ammonium groups comprise a C₁₋₂ alkyl group;

“Croquat® M” in which the quaternary ammonium groups comprise C₁₀-C₁₈ alkyl groups;

“Croquat® S” in which the quaternary ammonium groups comprise a C₁₋₈ alkyl group;

“Crotein® Q” in which the quaternary ammonium groups comprise at least one alkyl group comprising from 1 to 18 carbon atoms.

These various products are sold by the company Croda.

Other quaternized proteins or hydrolysates are, for example, those corresponding to the formula (XXII):

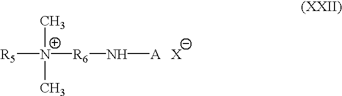

wherein X⁻ is an anion chosen from anions derived from organic and inorganic acids, A is a protein residue derived from hydrolysates of a protein, such as collagen, R₅ is chosen from lipophilic groups comprising up to 30 carbon atoms and R₆ is chosen from alkylene groups comprising 1 to 6 carbon atoms. Mention may be made, for example, of the products sold by the company Inolex under the name “Lexein® QX 3000”, referred to in the CTFA dictionary as “Cocotrimonium Collagen Hydrolysate”.

Mention may also be made, for example, of quaternized plant proteins such as wheat, corn and soybean proteins: as quaternized wheat proteins, mention may be made, for example, of those sold by the company Croda under the names “Hydrotriticum WQ or QM”, referred to in the CTFA dictionary as “Cocodimonium Hydrolysed Wheat Protein”, “Hydrotriticum QL”, referred to in the CTFA dictionary as “Lauridimonium Hydrolysed Wheat Protein” and “Hydrotriticum QS”, referred to in the CTFA dictionary as “Steardimonium Hydrolysed Wheat Protein”.

The cationic surfactants are, for example, chosen from quaternary ammonium salts, quaternary ammonium salts of imidazoline, diquaternary ammonium salts, and quaternary ammonium salts comprising at least one ester function.

The cationic surfactants may be chosen from: A)the quaternary ammonium salts of general formula (XXIII) below:

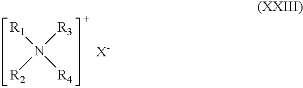

wherein X⁻ is an anion chosen from halides (chloride, bromide and iodide), (C₂-C₆)alkyl sulphates, such as methyl sulphate, phosphates, alkyl and alkylaryl sulphonates, and anions derived from organic acids, such as acetate and lactate, and

i) the radicals R₁ to R₃, which may be identical or different, are chosen from linear and branched aliphatic radicals comprising from 1 to 4 carbon atoms, and aromatic radicals such as aryl and alkylaryl. The aliphatic radicals can comprise at least one hetero atom such asoxygen, nitrogen, sulphur and halogens. The aliphatic radicals are chosen, for example, from alkyl, alkoxy and alkylamide radicals,

R₄ is chosen from linear and branched alkyl radicals comprising from 16 to 30 carbon atoms.

The cationic surfactant is, for example, a behenyltrimethylammonium salt (for example chloride).

ii) the radicals R₁ and R₂, which may be identical or different, are chosen from linear and branched aliphatic radicals comprising from 1 to 4 carbon atoms, and aromatic radicals such as aryl and alkylaryl. The aliphatic radicals can comprise at least one hetero atom such as oxygen, nitrogen, sulphur and halogens. The aliphatic radicals are chosen, for example, from alkyl, alkoxy, alkylamide and hydroxyalkyl radicals comprising from about 1 to 4 carbon atoms;

R₃ and R₄, which may be identical or different, are chosen from linear and branched alkyl radicals comprising from 12 to 30 carbon atoms, wherein the alkyl radicals may comprise at least one function chosen from ester and amide functions.

R₃ and R₄ are chosen, for example, from (C₁₂-C₂₂)alkylamido(C₂-C₆)alkyl and (C₁₂-C₂₂)alkylacetate radicals.

The cationic surfactant is, for example, a stearamidopropyldimethyl(myristyl acetate)ammonium salt (for example chloride);

B)—the quaternary ammonium salts of imidazolinium, such as that of formula (XXIV) below:

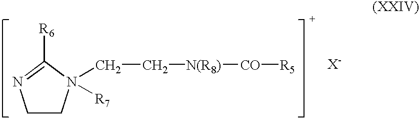

wherein R₅ is chosen from alkenyl and alkyl radicals comprising from 8 to 30 carbon atoms, for example fatty acid derivatives of tallow,

R₆ is chosen from a hydrogen atom, C₁-C₄ alkyl radicals and alkenyl and alkyl radicals comprising from 8 to 30 carbon atoms,

R₇ is chosen from C₁-C₄ alkyl radicals,

R₈ is chosen from a hydrogen atom and C₁-C₄ alkyl radicals, and

X⁻ is an anion chosen from halides, phosphates, acetates, lactates, alkyl sulphates, alkyl sulphonates and alkylaryl sulphonates.

In one embodiment, R₅ and R₆ are, for example, a mixture of radicals chosen from alkenyl and alkyl radicals comprising from 12 to 21 carbon atoms, such as fatty acid derivatives of tallow, R₇ is methyl and R₈ is hydrogen.

Such a product is, for example, Quaternium-27 (CTFA 1997) or Quaternium-83 (CTFA 1997), which are sold under the names “Rewoquat®” W75, W90, W75PG and W75HPG by the company Witco,

C)—the diquaternary ammonium salts of formula (XXV):

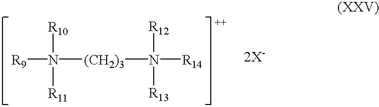

wherein

R₉ is chosen from aliphatic radicals comprising from 16 to 30 carbon atoms,

R₁₀, R₁₁, R₁₂, R₁₃ and R₁₄, which may be identical or different, are chosen from hydrogen and alkyl radicals comprising from 1 to 4 carbon atoms, and

X⁻ is an anion chosen from halides, acetates, phosphates, nitrates and methyl sulphates.

Such diquaternary ammonium salts, for example, include propanetallowdiammonium dichloride; and

D)—the quaternary ammonium salts comprising at least one ester function, of formula (XXVI) below:

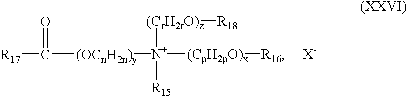

wherein:

R₁₅ is chosen from C₁-C₆ alkyl radicals and C₁-C₆ hydroxyalkyl and dihydroxyalkyl radicals;

R₁₆ is chosen from:

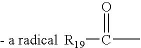

linear and branched, saturated and unsaturated C₁-C₂₂ hydrocarbon-based radicals R₂₀, and

a hydrogen atom,

R₁₈ is chosen from:

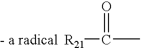

linear and branched, saturated and unsaturated C₁-C₆ hydrocarbon-based radicals R₂₂, and

a hydrogen atom,

R₁₇, R₁₉ and R₂₁, which may be identical or different, are chosen from linear and branched, saturated and unsaturated C₇-C₂₁ hydrocarbon-based radicals;

n, p and r, which may be identical or different, are chosen from integers ranging from 2 to 6;

y is chosen from integers ranging from 1 to 10;

x and z, which may be identical or different, are chosen from integers ranging from 0 to 10;

X⁻ is an anion chosen from simple and complex, organic and inorganic anions;

with the proviso that the sum x+y+z is from 1 to 15, that when x is 0, then R₁₆ is R₂₀ and that when z is 0, then R₁₈ is R₂₂.

In one embodiment, the ammonium salts of formula (XXVI) can be used,

wherein:

R₁₅ is chosen from methyl and ethyl radicals,

x and y are equal to 1;

z is equal to 0 or 1;

n, p and r are equal to 2;

R₁₆ is chosen from:

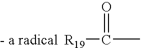

methyl, ethyl and C₁₄-C₂₂ hydrocarbon-based radicals, and

a hydrogen atom;

R₁₇, R₁₉ and R₂₁, which may be identical or different, are chosen from linear and branched, saturated and unsaturated C₇-C₂₁ hydrocarbon-based radicals;

R₁₈ is chosen from:

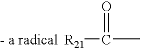

and

a hydrogen atom.

Such compounds are sold, for example, under the names “Dehyquart®” by the company Cognis, “Stepanquat®” by the company Stepan, “Noxamium®” by the company Ceca, and “Rewoquat® WE 18” by the company Rewo-Witco.

Among the quaternary ammonium salts examples are stearamidopropyldimethyl(myristyl acetate)ammonium chloride, sold under the name “Ceraphyl® 70” by the company Van Dyk, and Quaternium-27 or Quaternium-83 sold by the company Witco.

The cationic surfactants are classified in the category of soluble conditioners, but, depending on their chemical structures, they may be water-insoluble.

The at least one conditioner may be in an amount ranging from 0.001% to 20% by weight, for example, from 0.01% to 10% by weight and further, for example, from 0.1% to 3% by weight, relative to the total weight of the composition.

In one embodiment, the compositions comprise at least one entity chosen from cationic polymers andsilicones.

The compositions may also comprise at least one surfactant, which may be present in an amount ranging, for example, from 0.1% to 60% by weight, and further, for example, from 3% to 40% and even further, for example, from 5% to 30%, relative to the total weight of the composition.

The at least one surfactant may be chosen from anionic, amphoteric, nonionic and cationic surfactants.

The at least one surfactant that is suitable is, for example, chosen from:

(i) Anionic Surfactants:

In the context of the present disclosure, their nature is not critical.

As examples of anionic surfactants which can be used, alone or as mixtures, mention may be made, for example, of salts (such as alkaline salts, for example, sodium salts, ammonium salts, amine salts, amino alcohol salts and magnesium salts) of the following compounds: alkyl sulphates, alkyl ether sulphates, alkylamido ether sulphates, alkylarylpolyether sulphates, monoglyceride sulphates; alkyl sulphonates, alkyl phosphates, alkylamide sulphonates, alkylaryl sulphonates, α-olefin sulphonates, paraffin sulphonates; alkyl sulphosuccinates, alkyl ether sulphosuccinates, alkylamide sulphosuccinates; alkyl sulphosuccinamates; alkyl sulphoacetates; alkyl ether phosphates; acyl sarcosinates; acyl isethionates and N-acyltaurates. The alkyl or acyl radical of all of these various compounds, for example, comprises from 8 to 24 carbon atoms, and the aryl radical is chosen, for example, from phenyl and benzyl groups. Among the anionic surfactants which can also be used, mention may also be made of fatty acid salts such as the salts of oleic, ricinoleic, palmitic and stearic acids, coconut oil acid or hydrogenated coconut oil acid; acyl lactylates in which the acyl radical comprises from 8 to 20 carbon atoms. Weakly anionic surfactants can also be used, such as alkyl-D-galactosiduronic acids and their salts, as well as polyoxyalkylenated (C₆-C₂₄) alkyl ether carboxylic acids, polyoxyalkylenated (C₆-C₂₄) alkylaryl ether carboxylic acids, polyoxyalkylenated (C₆-C₂₄) alkylamido ether carboxylic acids and their salts, for example, those comprising from 2 to 50 ethylene oxide groups, and mixtures thereof.

Among the anionic surfactants, for example, alkyl sulphate salts and alkyl ether sulphate salts and mixtures thereof can be used.

(ii) Nonionic Surfactants:

The nonionic surfactants are compounds that are well known (see, for example, in this respect “Handbook of Surfactants” by M. R. Porter, published by Blackie & Son (Glasgow and London), 1991, pp. 116-178). In the present disclosure, the nature of the non-ionic surfactant is not critical. They can be chosen, for example, from polyethoxylated, polypropoxylated and polyglycerolated fatty acids, alkylphenols, α-diols and alcohols comprising a fatty chain comprising, for example, from 8 to 18 carbon atoms, it being possible for the number of ethylene oxide or propylene oxide groups to range, for example, from 2 to 50 and for the number of glycerol groups to range, for example, from 2 to 30. Mention may also be made of copolymers of ethylene oxide and of propylene oxide, condensates of ethylene oxide and of propylene oxide with fatty alcohols; polyethoxylated fatty amides, for example, comprising from 2 to 30 mol of ethylene oxide, polyglycerolated fatty amides comprising on average from 1 to 5, and such as from 1.5 to 4, glycerol groups; polyethoxylated fatty amines such as those containing from 2 to 30 mol of ethylene oxide; oxyethylenated fatty acid esters of sorbitan having from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides or N-acylaminopropylmorpholine oxides. It may be noted that the alkylpolyglycosides constitute nonionic surfactants that can be used.

(iii) Amphoteric Surfactant(s):

The amphoteric surfactants, whose nature is not critical, can be chosen, for example, from aliphatic secondary and tertiary amine derivatives in which the aliphatic radical is chosen from linear and branched chains comprising from 8 to 22 carbon atoms and comprising at least one water-soluble anionic group (for example carboxylate, sulphonate, sulphate, phosphate and phosphonate); mention may also be made of (C₈-C₂₀)alkylbetaines, sulphobetaines, (C₈-C₂₀)alkylamido(C₁-C₆)alkylbetaines or (C₈-C₂₀)alkylamido(C₁-C₆)alkylsulphobetaines.

Among the amine derivatives, mention may be made of the products sold under the name Miranol, as described, for example, in U.S. Pat. Nos. 2,528,378 and 2,781,354 and having the structures of:

R₂—CONHCH₂CH₂—N⁺(R₃)(R₄)(CH₂COO⁻) (XXVII)

wherein: R₂ is chosen from alkyl radicals derived from an acid R₂—COOH present in hydrolysed coconut oil, and heptyl, nonyl and undecyl radicals, R₃ is a P-hydroxyethyl group and R₄ is a carboxymethyl group; and of

R₅—CONHCH₂CH₂—N(B)(C) (XXVIII)

wherein B represents —CH₂CH₂OX′, C represents —(CH₂)′, —Y′, with z=1 or 2, X′ is chosen from the —CH₂CH₂—COOH group and a hydrogen atom, Y′ is chosen from —COOH and —CH₂—CHOH—SO₃H radicals, R₅ is chosen from alkyl radicals of an acid R₅—COOH present in coconut oil or in hydrolysed linseed oil, alkyl radicals, such as C₇, C₉, C₁₁ and C₁₋₃ alkyl radicals, a C₁₋₇ alkyl radical and its iso form, and an unsaturated C₁₇ radical.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, cocoamphodipropionic acid.

By way of example, mention may be made of the cocoamphodiacetate sold under the trade name Miranol C2M concentrate by the company Rhodia Chimie.

Among the anionic surfactants, sodium, triethanolamine and ammonium (C₁₂-C₁₄)alkyl sulphates, sodium, triethanolamine and ammonium (C₁₂-C₁₄)alkyl ether sulphates oxyethylenated with 2.2 mol of ethylene oxide, sodium cocoyl isethionate and sodium α-(C₁₄-C₁₆)olefin sulphonate, and mixtures thereof can be used, with:

either an amphoteric surfactant such as the amine derivatives known as disodium cocoamphodipropionate or sodium cocoamphopropionate sold, for example, by the company Rhodia Chimie under the trade name “Miranol® C2M CONC” as an aqueous solution comprising 38% active material, or under the name Miranol® C32;

or an amphoteric surfactant such as alkylbetaines, such as the cocobetaine sold under the name “Dehyton® AB 30” as an aqueous solution comprising 32% active material by the company Cognis, or (C₈-C₂₀)alkylamido(C₁-C₆)alkylbetaines, for example, Tegobetaine® F50 sold by the company Goldschmidt.

The composition may also comprise at least one additive chosen from thickeners, antidandruff and anti-seborrhoeic agents, fragrances, nacreous agents, hydroxy acids, electrolytes, preserving agents, silicone and non-silicone sunscreens, vitamins, provitamins such as panthenol, anionic and nonionic polymers, proteins, protein hydrolysates, 18-methyleicosanoic acid, and also mixtures of these various compounds and any other additive usually used in cosmetics that does not affect the properties of the compositions.

These additives are present in the composition in proportions that may range from 0 to 20% by weight relative to the total weight of the composition. The precise amount of each additive is readily determined by a person skilled in the art, depending on its nature and its function.

The compositions may be used, for example, for washing or treating keratin materials such as the hair, the skin, the eyelashes, the eyebrows, the nails, the lips or the scalp.

In one embodiment, the compositions are detergent compositions such as shampoos, shower gels and bubble baths. In this embodiment, the compositions comprise at least one washing base, which is generally aqueous.

The at least one washing base comprises at least one surfactant. The at least one surfactant may be chosen, without discrimination, alone or as mixtures, from the anionic, amphoteric and nonionic surfactants as defined above.

The quantity and quality of the washing base are those that are sufficient to be able to give the final composition satisfactory foaming power and/or detergent power.

Thus, the washing base can be in an amount ranging, for example, from 4% to 50% by weight, such as from 6% to 35% by weight and further such as from 8% to 25% by weight, relative to the total weight of the composition.

Another new embodiment is a process for treating a keratin material such as the skin or the hair, wherein the process comprises applying to the keratin material a cosmetic composition as defined above, and then optionally rinsing it out with water.

Thus, this process can allow the maintenance of the hairstyle and the treatment, care and washing or the removal of makeup from the skin, the hair or any other keratin material.

The compositions may also be in the form of rinse-out or leave-in conditioners, permanent-waving, hair-straightening, dyeing or bleaching compositions, or in the form of rinse-out compositions to be applied before or after dyeing, bleaching, permanent-waving or straightening the hair or between the two steps of a permanent-waving or hair-straightening operation.

When the composition is in the form of a conditioner, such as a rinse-out conditioner, it, for example, comprises at least one cationic surfactant, and its concentration generally ranges, for example, from 0.1% to 10% by weight, and such as from 0.5% to 5% by weight, relative to the total weight of the composition.

The compositions may also be in the form of washing compositions for the skin, such as in the form of bath or shower solutions or gels or makeup-removing products.

The compositions may also be in the form of aqueous or aqueous-alcoholic lotions for skincare and/or haircare.

The cosmetic compositions may be in the form of a gel, a milk, a cream, an emulsion, a thickened lotion or a mousse and may be used for the skin, the nails, the eyelashes, the lips and, for example, the hair.

The compositions may be packaged in various forms, such as in vaporizers, pump-dispenser bottles or in aerosol containers to allow the composition to be applied in vaporized form or in the form of a mousse. Such packaging forms are indicated, for example, when it is desired to obtain a spray, a lacquer or a mousse for treating a keratin material, such as the hair.

Throughout the text hereinabove and hereinbelow, the percentages expressed are on a weight basis.

New embodiments will now be illustrated more fully with the aid of the examples that follow, which cannot be considered as limiting it to the specific embodiments described.

In the examples, AM means active material.

### EXAMPLE 1

A shampoo having the composition below was prepared:

| | in |
|---|---|
| | g AM |
| Sodium lauryl ether sulphate (70/30 C12/C14) with 2.2 mol of | 7 |
| ethylene oxide, containing 70% AM | |
| Cocoylbetaine | 2.5 |
| Ethylene glycol distearate | 1.5 |
| Polydimethylsiloxane of formula (I) sold by Dow Corning under | 1.8 |
| the name DC2-8299 as a cationic emulsion containing 60% | |
| AM | |
| Polydimethylsiloxane of viscosity 60 000 cSt (DC200- | 1 |
| 60 000 cSt from Dow Corning) | |
| Hydroxyethylcellulose quaternized with 2,3- | 0.4 |
| epoxypropyltrimethylammonium chloride, sold under the name | |
| Ucare Polymer JR-400 by the company Amerchol | |
| Acrylic polymer in emulsion form, sold under the name Aqua | 0.8 |
| SF1 by Noveon | |
| Preserving agents | qs |
| pH agentsqs | pH 5.0 |
| Demineralized waterqs | 100 |

Hair treated with this shampoo has long-lasting softness and smoothness.

### EXAMPLE 2

A rinse-out conditioner having the composition below was prepared:

| Cetyl alcohol | | 3.7 | g |
|---|---|---|---|
| Myristyl alcohol | | 0.4 | g |
| Hydroxyethylcellulose (Natrosol 250 HHR from | | 0.25 | g |
| Aqualon) | | | |
| Mixture of myristyl/cetyl/stearyl | | 0.8 | g |
| myristate/palmitate/stearate (Blanc de Baleine | | | |
| Végétal from Laserson) | | | |
| Cetyltrimethylammonium chloride as an aqueous | | 2.5 | g |
| solution containing 25% AM (Dehyquart ® A OR | | (0.62 | g AM) |
| from Cognis) | | | |
| Behenyltrimethylammonium chloride as a | | 0.6 | g |
| water/isopropanol solution containing 80% AM | | (0.48 | g AM) |
| (Genamin KDM-F from Clariant) | | | |
| Polydimethylsiloxane of formula (I) sold by Dow | | 5 | g |
| Corning under the name DC2-8299 as a cationic | | (0.85 | g AM) |
| emulsion containing 60% AM | | | |
| Methosulphate (70/30 by weight) of | | 1 | g |
| dipalmitoylethylhydroxyethylmethylammonium/ | | | |
| cetyl alcohol/stearyl alcohol (Dehyquart F 30 from | | | |
| Cognis) | | | |
| Citric acid | qs | pH 3.5 ± 0.5 | |
| Fragrance, preserving agents, | qs | | |
| Deionized water | qs | 100 | g |

Hair treated with this composition has long-lasting softness and smoothness.

### EXAMPLE 3

| Composition | Example 6 | |
|---|---|---|
| Sodium lauryl ether sulphate containing 2.2 mol of | 16 | g AM |
| ethylene oxide | | |
| Cocoylbetaine | 2.4 | g AM |
| Methacrylic acid/ethyl acrylate crosslinked | 0.8 | g AM |
| copolymer as an aqueous emulsion containing | | |
| 30% AM, sold under the name Carbopol Aqua SF1 | | |
| by the company Noveon | | |
| Polydimethylsiloxane of viscosity 60 000 cSt, sold | 1 | g |
| under the name DC200 fluid 60 000 cSt by the | | |
| company Dow Corning | | |
| Polydimethylsiloxane containing aminoethyl | 1.8 | g AM |
| iminobutyl groups, as a cationic 60% emulsion in | | |
| water, sold under the name DC2-8299 by the | | |
| company Dow Corning | | |
| Dimethyl diallyl ammonium chloride homopolymer | 0.4 | g |
| as a 40% AM aqueous emulsion sold under the | | |
| name MERQUAT 100 by the company NALCO | | |
| Behenylic alcohol | 1.5 | g |
| Distearylether | 1.5 | g |
| Preserving agents | | qs |
| Citric acid or sodium hydroxideqs | pH | 7 |
| Demineralized waterqs | 100 | g |

Hair treated with this shampoo has long-lasting softness and smoothness.

What is claimed is:

1. A cosmetic composition comprising, in a cosmetically acceptable medium, at least one aminosilicone comprising at least one aminoethylimino(C₄-C₈)alkyl group and at least one conditioner.

2. The composition according to claim 1, wherein the at least one aminosilicone is of formula (I) below: 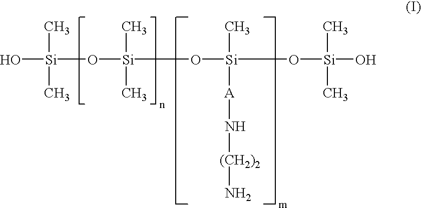
in which:
m and n are numbers such that the sum (n+m) ranges from 1 to 2000, n is a number ranging from 0 to 1999, and m is a number ranging from 1 to 2000; and
A is chosen from linear and branched alkylene radicals comprising from 4 to 8 carbon atoms.

3. The composition according to claim 2, wherein the sum (n+m) ranges from 50 to 150.

4. The composition according to claim 2, wherein the n is a number ranging from 49 to 149.

5. The composition according to claim 2, wherein the m is a number ranging from 1 to 10.

6. The composition according to claim 2, wherein the A is chosen from linear and branched alkylene radicals comprising 4 carbon atoms.

7. The composition according to claim 1, wherein the viscosity of the at least one aminosilicone is greater than 25 000 cSt (mm²/s) at 25° C.

8. The composition according to claim 7, wherein the viscosity of the at least one aminosilicone ranges from 30 000 to 200 000 cSt(mm²/s) at 25° C.

9. The composition according to claim 1, wherein the weight-average molecular mass of the at least one aminosilicone ranges from 2000 to 1 000 000.

10. The composition according to claim 9, wherein the weight-average molecular mass of the at least one aminosilicone ranges from 3500 to 200 000.

11. The composition according to claim 1, wherein the at least one aminosilicone is in the form of an oil-in-water emulsion.

12. The composition according to claim 11, wherein the oil-in-water emulsion comprises at least one surfactant chosen from cationic and nonionic surfactants.

13. The composition according to claim 11, wherein the number-average particle size of the at least one aminosilicone in the emulsion ranges from 3 to 500 nanometres.

14. The composition according to claim 13, wherein the number-average particle size of the at least one aminosilicone in the emulsion ranges from 5 to 300 nanometres.

15. The composition according to claim 14, wherein the number-average particle size of the at least one aminosilicone in the emulsion ranges from 10 to 275 nanometres.

16. The composition according to claim 1, wherein the at least one aminosilicone is present in a concentration ranging from 0.01% to 20% by weight relative to the total weight of the composition.

17. The composition according to claim 16, wherein the at least one aminosilicone is present in a concentration ranging from 0.1% to 15% by weight relative to the total weight of the composition.

18. The composition according to claim 17, wherein the at least one aminosilicone is present in a concentration ranging from 0.5% to 10% by weight relative to the total weight of the composition.

19. The composition according to claim 1, wherein the at least one conditioner is chosen from synthetic oils, mineral oils, plant oils, fluoro oils and perfluoro oils, natural and synthetic waxes, compounds of ceramide type, carboxylic acid esters, silicones other than those comprising at least one aminoethylimino(C₄-C₈)alkyl group, anionic polymers, nonionic polymers, cationic polymers, amphoteric polymers, cationic proteins, cationic protein hydrolysates and cationic surfactants.

20. The composition according to claim 19, wherein the synthetic oils are chosen from polyolefins of hydrogenated and non-hydrogenated polybutene types and polyolefins of hydrogenated and non-hydrogenated polydecene types.

21. The composition according to claim 19, wherein the plant oils are chosen from sunflower oil, corn oil, soybean oil, avocado oil, jojoba oil, marrow oil, grapeseed oil, sesame oil, hazelnut oil, fish oils, glyceryl tricaprocaprylate, and plant and animal oils of formula R₉COOR₁₀ in which R₉ is chosen from higher fatty acid residues comprising from 7 to 29 carbon atoms and R₁₀ is chosen from linear and branched hydrocarbon-based chains comprising from 3 to 30 carbon atoms and natural and synthetic essential oils.

22. The composition according to claim 21, wherein R₁₀ is chosen from alkyl and alkenyl radicals.

23. The composition according to claim 21, wherein the natural and synthetic essential oils are chosen from eucalyptus oil, lavendin oil, lavender oil, vetiver oil, Litsea cubeba oil, lemon oil, sandalwood oil, rosemary oil, camomile oil, savory oil, nutmeg oil, cinnamon oil, hyssop oil, caraway oil, orange oil, geraniol oil, cade oil and bergamot oil.

24. The composition according to claim 19, wherein the natural and synthetic waxes are chosen from carnauba wax, candelilla wax, alfalfa wax, paraffin wax, ozokerite, plant waxes, absolute waxes of flowers, animal waxes, marine waxes, polyethylene waxes and polyolefins.

25. The composition according to claim 24, wherein the plant waxes are chosen from olive tree wax, rice wax, and hydrogenated jojoba wax.

26. The composition according to claim 24, wherein the absolute waxes of flowers are chosen from essential waxes of blackcurrant flowers.

27 The composition according to claim 24, wherein the animal waxes are beeswaxes.

28. The composition according to claim 19, wherein the compounds of ceramide type are chosen from:
2-N-linoleoylaminooctadecane-1,3-diol,
2-N-oleoylaminooctadecane-1,3-diol,
2-N-palmitoylaminooctadecane-1,3-diol,
2-N-stearoylaminooctadecane-1,3-diol,
2-N-behenoylaminooctadecane-1,3-diol,
2-N-[2-hydroxypalmitoyl]aminooctadecane-1,3-diol,
2-N-stearoylaminooctadecane-1,3,4-triol,
2-N-palmitoylaminohexadecane-1,3-diol,
bis(N-hydroxyethyl-N-cetyl)malonamide,
N-(2-hydroxyethyl)-N-(3-cetyloxy-2-hydroxypropyl)cetylamide,
N-docosanoyl-N-methyl-D-glucamine,
and mixtures thereof.

29. The composition according to claim 28, wherein the 2-N-stearoylaminooctadecane-1,3,4-triol is N-stearoylphytosphingosine.

30. The composition according to claim 19, wherein the carboxylic acid esters are chosen from ethyl and isopropyl palmitates, 2-ethylhexyl palmitate, 2-octyldecyl palmitate, alkyl myristates, hexyl stearate, butyl stearate, isobutyl stearate, dioctyl malate, hexyl laurate, 2-hexyldecyl laurate, isononyl isononanoate and cetyl octanoate.

31. The composition according to claim 30, wherein the alkyl myristates are chosen from isopropyl, butyl, cetyl and 2-octyidodecyl myristates.

32. The composition according to claim 19, wherein the silicones other than those comprising at least one aminoethylimino(C₄-C₈)alkyl group are non-volatile polyorganosiloxanes chosen from polyalkylsiloxanes, polyarylsiloxanes, polyalkylarylsiloxanes, silicone gums and silicone resins, polyorganosiloxanes modified with organofunctional groups, and mixtures thereof.

33. The composition according to claim 32, wherein:
(a) the polyalkylsiloxanes are chosen from:
polydimethylsiloxanes comprising trimethylsilyl end groups;
polydimethylsiloxanes comprising dimethylsilanol end groups; and
poly(C₁-C₂₀)alkylsiloxanes;
(b) the polyalkylarylsiloxanes are chosen from:
linear and branched polydimethylmethylphenylsiloxanes; and polydimethyldiphenylsiloxanes with a viscosity ranging from 1×10⁻⁵ to 5×10⁻² m²/s at 25° C.;
(c) the silicone gums are chosen from polydiorganosiloxanes with number-average molecular masses ranging from 200 000 to 1 000 000, used alone or in the form of a mixture in a solvent;
(d) the silicone resins are chosen from resins comprising at least one unit chosen from units of R₃SiO_{1/2}, R₂SiO_{2/2}, RSiO_{3/2} and SiO_{4/2}
in which R is chosen from hydrocarbon-based groups comprising from 1 to 16 carbon atoms and a phenyl group;
(e) the polyorganosiloxanes are chosen from silicones comprising at least one organofunctional group attached via a hydrocarbon-based radical.

34. The composition according to claim 33, wherein the silicone gums are chosen from:
polydimethylsiloxane
polydimethylsiloxane/methylvinylsiloxane,
polydimethylsiloxane/diphenylsiloxane,
polydimethylsiloxane/phenylmethylsiloxane,
polydimethylsiloxane/diphenylsiloxane/methylvinylsiloxane, and the following mixtures:
mixtures formed from a polydimethylsiloxane hydroxylated at the end of the chain and from a cyclic polydimethylsiloxane;
mixtures formed from a polydimethylsiloxane gum and from a cyclic silicone; and
mixtures of polydimethylsiloxanes of different viscosities.

35. The composition according to claim 32, wherein the polyorganosiloxanes modified with organofunctional groups are chosen from polyorganosiloxanes comprising at least one group chosen from:
a) polyethyleneoxy and polypropyleneoxy groups;
b) substituted and unsubstituted amine groups;
c) thiol groups;
d) alkoxylated groups;
e) hydroxyalkyl groups;
f) acyloxyalkyl groups;
g) alkylcarboxylic groups;
h) 2-hydroxyalkylsulphonate groups;
i) 2-hydroxyalkylthiosulphonate groups; and
j) hydroxyacylamino groups.

36. The composition according to claim 32, wherein the non-volatile polyorganosiloxanes are chosen from polyalkylsiloxanes comprising trimethylsilyl end groups, polyalkylsiloxanes comprising dimethylsilanol end groups, polyalkylarylsiloxanes, mixtures of two PDMSs comprising a gum and an oil of different viscosities, mixtures of organosiloxanes and of cyclic silicones, and polyorganosiloxane resins.

37. The composition according to claim 19, wherein the anionic polymers are chosen from:
polymers comprising carboxylic units derived from monomers chosen from unsaturated mono- and dicarboxylic acid monomers of the following formula: 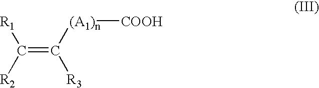
in which n is an integer ranging from 0 to 10, A₁ is a methylene group, optionally connected to the carbon atom of the unsaturated group, or to the neighbouring methylene group when n is greater than 1, via a hetero atom, R₁ is chosen from a hydrogen atom and phenyl and benzyl groups, R₂ is chosen from a hydrogen atom and lower alkyl and carboxyl groups, R₃ is chosen from a hydrogen atom, lower alkyl groups and —CH₂—COOH, phenyl and benzyl groups;
polymers comprising units derived from sulphonic acids.

38. The composition according to claim 37, wherein in defining A₁, the hetero atom is chosen from oxygen and sulphur.

39. The composition according to claim 37, wherein the units derived from sulphonic acids are chosen from vinylsulphonic, styrenesulphonic and acrylamidoalkylsulphonic units.

40. The composition according to claim 19, wherein the anionic polymers are chosen from:
acrylic acid copolymers;
copolymers derived from crotonic acid;
polymers derived from entities chosen from maleic, fumaric and itaconic acids and anhydrides with vinyl esters, vinyl ethers, vinyl halides and phenylvinyl derivatives, acrylic acid and its esters;
copolymers of methacrylic acid and of methyl methacrylate;
the copolymer of methacrylic acid and of ethyl acrylate;
vinyl acetate/crotonic acid copolymer; and
vinyl acetate/crotonic acid/polyethylene glycol terpolymer.

41. The composition according to claim 40, wherein the acrylic acid copolymers are acrylic acid/ethyl acrylate/N-tert-butylacrylamide terpolymers.

42. The composition according to claim 40, wherein the copolymers derived from crotonic acid are chosen from vinyl acetate/vinyl tert-butylbenzoate/crotonic acid terpolymers and crotonic acid/vinyl acetate/vinyl neododecanoate terpolymers.

43. The composition according to claim 40, wherein the polymers derived from the entities are methyl vinyl ether/monoesterified maleic anhydride copolymers.

44. The composition according to claim 19, wherein the amphoteric polymers are chosen from polymers comprising at least one unit derived from:
a) at least one monomer chosen from acrylamides and methacrylamides substituted on the nitrogen with a radical chosen from alkyl radicals,
b) at least one acidic comonomer comprising at least one reactive carboxylic group, and
c) at least one basic comonomer.

45. The composition according to claim 44, wherein in (c) the at least one basic comonomer is chosen from esters comprising primary, secondary, tertiary and quaternary amine substituents of acrylic and methacrylic acids and the product of quaternization of dimethylaminoethyl methacrylate with a sulphate chosen from dimethyl and diethyl sulphates.

46. The composition according to claim 19, wherein the nonionic polymers are chosen from:
polyalkyloxazolines;
vinyl acetate homopolymers;
copolymers of vinyl acetate and acrylic ester;
copolymers of vinyl acetate and ethylene;
copolymers of vinyl acetate and maleic ester;
copolymers of polyethylene and maleic anhydride;
alkyl acrylate homopolymers and alkyl methacrylate homopolymers;
acrylic ester copolymers;
copolymers of acrylonitrile and a nonionic monomer chosen; and
copolymers of alkyl acrylate and of urethane.

47. The composition according to claim 46, wherein the acrylic ester copolymers are chosen from copolymers of alkyl acrylates and alkyl methacrylates.

48. The composition according to claim 46, wherein the nonionic monomer is chosen from butadiene and alkyl (meth)acrylates.

49. The composition according to claim 19, wherein the cationic polymers are chosen from polymers comprising units comprising at least one amine group chosen from primary, secondary, tertiary and quaternary amine groups that either form part of the main polymer chain or are borne by a side substituent directly attached to the main polymer chain.

50. The composition according to claim 49, wherein the cationic polymers are chosen from cationic cyclopolymers, cationic polysaccharides, and quaternary polymers of vinylpyrrolidone and of vinylimidazole, and mixtures thereof.

51. The composition according to claim 50, wherein the cationic cyclopolymers are chosen from diallyidimethylammonium chloride homopolymers and copolymers of diallyldimethylammonium chloride and of acrylamide.

52. The composition according to claim 50, wherein the cationic polysaccharides are chosen from hydroxyethylcelluloses that have reacted with an epoxide substituted with a trimethylammonium group.

53. The composition according to claim 50, wherein the cationic polysaccharides are chosen from guar gums modified with a 2,3-epoxypropyltrimethylammonium salt.

54. The composition according to claim 19, wherein the cationic surfactants are chosen from:
A) the quaternary ammonium salts of general formula (XXIII) below: 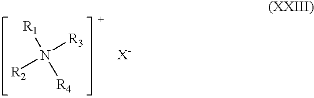
wherein X⁻ is an anion chosen from halides, (C₂-C₆)alkyl sulphates, phosphates, alkyl and alkylaryl sulphonates, and anions derived from organic acids, and
i) the radicals R₁, R₂, and R₃, which may be identical or different, are chosen from linear and branched aliphatic radicals comprising from 1 to 4 carbon atoms, and aromatic radicals; R₄ is chosen from linear and branched alkyl radicals comprising from 16 to 30 carbon atoms; or
ii) the radicals R₁ and R₂, which may be identical or different, are chosen from linear and branched aliphatic radicals comprising from 1 to 4 carbon atoms, and aromatic radicals;
R₃ and R₄, which may be identical or different, are chosen from linear and branched alkyl radicals comprising from 12 to 30 carbon atoms, wherein the alkyl radicals comprise at least one function chosen from ester and amide functions;
B)—the quaternary ammonium salts of imidazolinium;
C)—the diquaternary ammonium salts of formula (XXV): 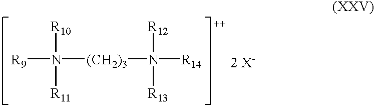
in which
R₉ is chosen from aliphatic radicals comprising from 16 to 30 carbon atoms,
R₁₀, R₁₁, R₁₂, R₁₃ and R₁₄, which may be identical or different, are chosen from hydrogen and alkyl radicals comprising from 1 to 4 carbon atoms, and X⁻ is an anion chosen from halides, acetates, phosphates, nitrates and methyl sulphates;
D)—the quaternary ammonium salts comprising at least one ester function, of formula (XXVI) below: 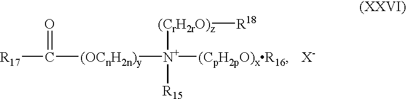
in which:
R₁₅ is chosen from C₁-C₆ alkyl radicals and C₁-C₆ hydroxyalkyl and dihydroxyalkyl radicals;
R₁₆ is chosen from:
a radical 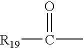
linear and branched, saturated and unsaturated C₁-C₂₂ hydrocarbon-based radicals R₂₀, and
a hydrogen atom,
R₁₈ is chosen from:
a radical 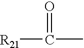
linear and branched, saturated and unsaturated C₁-C₆ hydrocarbon-based radicals R₂₂, and
a hydrogen atom,
R₁₇, R₁₉ and R₂₁, which may be identical or different, are chosen from linear and branched, saturated and unsaturated C₇-C₂₁ hydrocarbon-based radicals;
n, p and r, which may be identical or different, are chosen from integers ranging from 2 to 6;
y is chosen from integers ranging from 1 to 10;
x and z, which may be identical or different, are chosen from integers ranging from 0 to 10;
X⁻ is a an anion chosen from simple and complex, organic and inorganic anions; with the proviso that the sum x+y+z is from 1 to 15, that when x is 0, then R₁₆ is R₂₀ and that when z is 0, then R₁₈ is R₂₂.

55. The composition according to claim 54, wherein in A) the halides are chosen from chloride, bromide and iodide.

56. The composition according to claim 54, wherein in A) the (C₂-C₆)alkyl sulphates are a methyl sulphate.

57. The composition according to claim 54, wherein in A) the anions derived from organic acids are chosen from acetates and lactates.

58. The composition according to claim 54, wherein in A) the aromatic radicals are chosen from aryl and alkylaryl radicals.

59. The composition according to claim 54, wherein in A) the aliphatic radicals optionally comprise at least one hetero atom.

60. The composition according to claim 59, wherein the at least one hetero atom is chosen from oxygen, nitrogen, sulphur and halogens

61. The composition according to claim 59, wherein in A) the aliphatic radicals are chosen from alkyl, alkoxy and alkylamide radicals.

62. The composition according to claim 54, wherein, in A) ii) the R₃ and R₄ are chosen from (C₁₂-C₂₂)alkylamido(C₂-C₆)alkyl and (C₁₂-C₂₂)alkylacetate radicals.

63. The composition according to claim 54, wherein, in A) the cationic surfactant is a stearamidopropyldimethyl(myristyl acetate)ammonium salt.

64. The composition according to claim 63, wherein the stearamidopropyldimethyl(myristyl acetate)ammonium salt is stearamidopropyldimethyl(myristyl acetate)ammonium chloride.

65. The composition according to claim 54, wherein in B) the quaternary ammonium salts of imidazolinium are chosen from those of formula (XXIV) below: 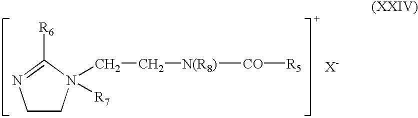
in which
R₅ is chosen from alkenyl and alkyl radicals comprising from 8 to 30 carbon atoms,
R₆ is chosen from a hydrogen atom, C₁-C₄ alkyl radicals and alkenyl and alkyl radicals comprising from 8 to 30 carbon atoms,
R₇ is chosen from C₁-C₄ alkyl radicals,
R₈ is chosen from a hydrogen atom and C₁-C₄ alkyl radicals, and
X⁻ is an anion chosen from halides, phosphates, acetates, lactates, alkyl sulphates, alkyl sulphonates and alkylaryl sulphonates.

66. The composition according to claim 65, wherein R₅ is chosen from fatty acid derivatives of tallow.

67. The composition according to claim 65, wherein R₅ and R₆ are chosen from mixtures of radicals chosen from alkenyl and alkyl radicals comprising from 12 to 21 carbon atoms,
R₇ is a methyl radical, and
R₈ is a hydrogen atom.

68. The composition according to claim 67, wherein R₅ and R₆ are chosen from mixtures of fatty acid derivatives of tallow.

69. The composition according to claim 65, wherein the quaternary ammonium salts of imidazolinium are chosen from Quaternium-27 (CTFA 1997) and Quaternium-83 (CTFA 1997).

70. The composition according to claim 54, wherein in C) the diquaternary ammonium salts are a propanetallowdiammonium dichloride.

71. The composition according to claim 1, wherein the at least one conditioner is present in a concentration ranging from 0.001% to 20% by weight relative to the total weight of the composition.

72. The composition according to claim 71, wherein the at least one conditioner is present in a concentration ranging from 0.01% to 10% by weight relative to the total weight of the composition.

73. The composition according to claim 1, further comprising at least one entity chosen from cationic polymers and silicones.

74. The composition according to claim 1, further comprising at least one surfactant chosen from anionic, nonionic, amphoteric and cationic surfactants.

75. The composition according to claim 74, wherein the at least one surfactant is present in a concentration ranging from 0.1% to 60% by weight relative to the total weight of the composition.

76. The composition according to claim 75, wherein the at least one surfactant is present in a concentration ranging from 3% and 40% by weight relative to the total weight of the composition.

77. The composition according to claim 76, wherein the at least one surfactant is present in a concentration ranging from 5% and 30% by weight relative to the total weight of the composition.

78. The composition according to claim 1, further comprising at least one additive chosen from thickeners, antidandruff and anti-seborrhoeic agents, fragrances, nacreous agents, hydroxy acids, electrolytes, preserving agents, silicone and non-silicone sunscreens, vitamins, provitamins, proteins, protein hydrolysates, 18-methyleicosanoic acid and panthenol.

79. The composition according to claim 1, wherein the composition is in a form chosen from shampoos, conditioners, compositions for permanent-waving, straightening, dyeing or bleaching hair, rinse-out compositions to be applied between the two steps of a permanent-waving or hair-straightening operation, and washing compositions for a body.

80. A composition for washing or caring for a keratin material comprising, in a cosmetically acceptable medium, at least one aminosilicone comprising at least one aminoethylimino(C₄-C₈)alkyl group and at least one conditioner, wherein the composition is effective for washing or caring for the keratin material.

81. A method for washing or caring for a keratin material comprising applying to the keratin material a composition comprising, in a cosmetically acceptable medium, at least one aminosilicone comprising at least one aminoethylimino(C₄-C₈)alkyl group and at least one conditioner.

82. A method of manufacturing a cosmetic composition comprising including in the composition at least one aminosilicone comprising at least one aminoethylimino(C₄-C₈)alkyl group and at least one conditioner

83. A composition for conditioning a keratin material comprising, in a cosmetically acceptable medium, at least one aminosilicone comprising at least one aminoethylimino(C₄-C₈)alkyl group and at least one conditioner, wherein the composition is effective for conditioning the keratin material.

84. A process for conditioning a keratin material comprising applying to the keratin material a composition comprising, in a cosmetically acceptable medium, at least one aminosilicone comprising at least one aminoethylimino(C₄-C₈)alkyl group and at least one conditioner.

85. A process for treating a keratin material comprising applying to the keratin material a cosmetic composition comprising, in a cosmetically acceptable medium, at least one aminosilicone comprising at least one aminoethylimino(C₄-C₈)alkyl group and at least one conditioner, and then optionally rinsing the keratin material with water.

86. The process according to claim 85, wherein the keratin material is hair.

87. A composition for improving lightness, softness, sheen and/or disentangling, and/or providing ease of styling of a keratin material comprising, in a cosmetically acceptable medium, at least one aminosilicone comprising at least one aminoethylimino(C₄-C₈)alkyl group and at least one conditioner;
wherein the composition is effective in improving lightness, softness, sheen and/or disentangling, and/or providing ease of styling of the keratin material.

88. A method for improving lightness, softness, sheen and/or disentangling, and/or providing ease of styling of a keratin material comprising applying to the keratin material a composition comprising, in a cosmetically acceptable medium, at least one aminosilicone comprising at least one aminoethylimino(C₄-C₈)alkyl group and at least one conditioner.

89. A composition for improving persistent and remanent effects with respect to shampooing several times comprising, in a cosmetically acceptable medium, at least one aminosilicone comprising at least one aminoethylimino(C₄-C₈)alkyl group and at least one conditioner; wherein the composition is effective for improving persistent and remanent effects with respect to shampooing several times.

90. A method for improving persistent and remanent effects with respect to shampooing several times, comprising including in a composition at least one aminosilicone comprising at least one aminoethylimino(C₄-C₈)alkyl group and at least one conditioner.

91. A composition for improving remanence of the conditioning effects with respect to shampooing comprising, in a cosmetically acceptable medium, at least one aminosilicone comprising at least one aminoethylimino(C₄-C₈)alkyl group and at least one conditioner; wherein the composition is effective in improving remanence of the conditioning effects with respect to shampooing.

92. A method for improving remanence of the conditioning effects with respect to shampooing, comprising including in a composition at least one aminosilicone comprising at least one aminoethylimino(C₄-C₈)alkyl group and at least one conditioner.

93. A process for improving remanence of conditioning effects of a cosmetic composition with respect to shampooing comprising including in said composition at least one aminosilicone, wherein the at least one aminosilicone comprises at least one aminoethylimino(C₄-C₈)alkyl group.
